(19) United States
(12) Patent Application Publication
(10) Pub. No.: US 2020/0316268 A1
(43) Pub. Date: Oct. 8, 2020

(54) IMMOBILISING BIOLOGICAL ENTITIES

(71) Applicant: CARMEDA AB, Upplands Väsby (SE)

(72) Inventors: Per ANTONI, Upplands Väsby (SE); Malin ERIKSSON, Upplands Väsby (SE); Anna GÄLLHAGEN, Upplands Väsby (SE); Eva KOCH, Upplands Väsby (SE); Daniel NYSTRÖM, Upplands Väsby (SE); Christian PORSCH-GRAHM, Upplands Väsby (SE); Helena GÖRANSSON, Upplands Väsby (SE); Peter GUNNARSSON, Upplands Väsby (SE)

(21) Appl. No.: 16/841,125

(22) Filed: Apr. 6, 2020

Related U.S. Application Data
(63) Continuation of application No. 16/296,906, filed on Mar. 8, 2019.

(30) Foreign Application Priority Data
Mar. 9, 2018 (EP) 18161066.8

(51) Int. Cl. A61L 33/00; A61L 29/16; A61L 29/08; A61L 33/08; A61L 33/06

(52) U.S. Cl. CPC A61L 33/0035; A61L 29/16; A61L 29/085; A61L 33/0029; A61L 2420/08; A61L 33/068; A61L 33/0076; A61L 2300/42; A61L 2420/02; A61L 33/08

(57) ABSTRACT

There is provided inter alia a solid object having a surface comprising a layered coating of cationic and anionic polymer, wherein the outer coating layer is a layer comprising cationic polymer to which is covalently bound an anticoagulant entity; and wherein the anionic polymer is characterized by having (a) a total molecular weight of 20 kDa-650 kDa; and (b) a solution charge density of ≤4 μeq/g.

### FIELD OF THE INVENTION

The present invention relates to solid objects having surface coatings comprising biological entities, and to processes for preparing such surface coatings. In particular, the present invention relates to improved surface coatings comprising anticoagulant entities such as heparin, and to processing for preparing such surface coatings.

### BACKGROUND OF THE INVENTION

When a medical device is implanted in the body or is in contact with body fluids, a number of different reactions are set into motion, some of them resulting in inflammation and some in the coagulation of the blood in contact with the device surface. In order to counteract these serious adverse effects, the well-known anticoagulant compound heparin has for a long time been administered systemically to patients before the medical device is implanted into their body, or when it is in contact with their body fluids, in order to provide an antithrombotic effect.

Thrombin is one of several coagulation factors, all of which work together to result in the formation of thrombi at a surface in contact with the blood. Antithrombin (also known as antithrombin III) (“ATIII”) is the most prominent coagulation inhibitor. It neutralizes the action of thrombin and other coagulation factors and thus restricts or limits blood coagulation. Heparin dramatically enhances the rate at which antithrombin inhibits coagulation factors. Heparin cofactor II (“HCII”) is another coagulation factor which rapidly inhibits thrombin in the presence of heparin.

Systemic treatment with high doses of heparin is, however, often associated with serious side-effects of which bleeding is the predominant. Another rare, but serious complication of heparin therapy is the development of an allergic response called heparin induced thrombocytopenia (HIT) that may lead to thrombosis (both venous and arterial). High-dose systemic heparin treatment e.g. during surgery also requires frequent monitoring of the activated clotting time (used to monitor and guide heparin therapy) and the corresponding dose adjustments as necessary.

Therefore, solutions have been sought where the need for a systemic heparinization of the patient would be unnecessary or can be limited. It was thought that this could be achieved through a surface modification of the medical devices using the anticoagulative properties of heparin and other anticoagulants. Thus, a number of more or less successful technologies have been developed where a layer of heparin is attached to the surface of the medical device seeking thereby to render the surface thromboresistant. For devices where long-term bioactivity is required, heparin should desirably be resistant to leaching and degradation.

Heparin is a polysaccharide carrying negatively charged sulfate and carboxylic acid groups on the saccharide units. Ionic binding of heparin to polycationic surfaces was thus attempted, but the surface modifications suffered from lack of stability resulting in lack of function, as the heparin leached from the surface. Thereafter, different surface modifications have been prepared wherein the heparin has been covalently bound to groups on the surface.

One of the most successful processes for rendering a medical device thromboresistant has been the covalent binding of a heparin fragment to a modified surface of the device. The general method and improvements thereof are described in various patent documents (see EP0086186A1, EP0086187A1, EP0495820B1 and U.S. Pat. No. 6,461,665B1 each of which is incorporated herein by reference in its entirety).

These documents describe the preparation of a heparinized surface by reacting heparin modified to bear a terminal aldehyde group with a surface on a medical device which has been modified to bear primary amino groups. An intermediate Schiff base is formed which is reduced in situ to form a stable secondary amine linker, thereby covalently immobilizing the heparin.

Further methods for covalently attaching heparin to a surface while retaining its activity are described in WO2010/029189A2, WO2011/110684A1 and WO2012/123384A1 (each of which is incorporated herein by reference in its entirety).

The anticoagulant entity is typically immobilized on a surface which has been treated with one or more layers of polymer or a complex, rather than being immobilized directly onto the surface of the solid object.

EP0086187A1 describes a surface modified substrate with a complex absorbed thereto, wherein the complex is of a polymeric cationic surfactant that contains primary amino nitrogen functionality as well as secondary and/or tertiary amino functionality, and a dialdehyde that has 1-4 carbon atoms between the two aldehyde groups. An anionic compound may additionally be bonded to said complex, and optionally additional cationic and anionic alternating compounds.

EP0495820B1 describes a method for modifying the surface of a substrate, comprising the steps of: (a) adsorbing a polyamine of a high average molecular weight and crosslinking said polyamine with crotonaldehyde; (b) then adsorbing on the surface of the crosslinked polyamine a layer of an anionic polysaccharide; (c) optionally repeating steps (a) and (b) one or more times; and (d) adsorbing on the anionic polysaccharide layer, or on the outermost layer of anionic polysaccharide, a layer of non-crosslinked polyamine providing free primary amino groups. In a subsequent step, a biologically active chemical entity carrying a functional group reactive with the free primary amino groups can be bound to the non-crosslinked polyamine, e.g. heparin.

However, there remains a need for improved surface coatings comprising anticoagulant entities such as heparin, in particular for coatings in which the biological activity of the anticoagulant entity is maintained or enhanced. Such improved surface coatings have potential utility in medical devices and other articles which would benefit from an anticoagulant surface.

The present inventors have discovered that, surprisingly, the nature of the surface upon which an anticoagulant entity is immobilized can significantly impact characteristics of the coating, in particular the resulting biological activity of the anticoagulant entity. In particular, when an anticoagulant entity is immobilized on a surface of a solid object comprising a layered coating of cationic and anionic polymer, careful modulation of the nature and the conditions of the application of the anionic polymer layer(s) can improve the resulting characteristics of the coating of the solid object including, for example, the thromboresistant properties that it may have.

### SUMMARY OF THE INVENTION

In one aspect, the present invention provides a solid object having a surface comprising a layered coating of cationic and anionic polymer, wherein the outer coating layer is a layer comprising cationic polymer to which is covalently bound an anticoagulant entity; and wherein the anionic polymer is characterized by having (a) a total molecular weight of 20-650 kDa; and (b) a solution charge density of ≤4 μeq/g.

In another aspect, the present invention provides a process for the manufacture of a solid object having a surface comprising a layered coating of cationic and anionic polymer, wherein the outer coating layer comprises cationic polymer to which is covalently bound an anticoagulant entity, comprising the steps of:

i) treating a surface of the solid object with a cationic polymer;

ii) treating the surface with an anionic polymer;

iii) optionally repeating steps i) and ii) one or more times;

iv) treating the surface with a cationic polymer; and

v) treating the outermost layer of cationic polymer with an anticoagulant entity, thereby to covalently attach the anticoagulant entity to the outermost layer of cationic polymer; wherein, the anionic polymer is characterized by having (a) a total molecular weight of 20 kDa-650 kDa; and (b) a solution charge density of ≤4 μeq/g.

### BRIEF DESCRIPTION OF THE FIGURES

FIG. 1: shows an example coating of the invention with a single bilayer;

FIG. 2: shows preserved platelets (%) for PVC tubing coated with dextran sulfates 1, 2, 4 and 6 at 1.7 M NaCl concentration;

FIG. 3: shows F1+2 (prothrombin fragment) for PVC tubing coated with dextran sulfates 1, 2, 4 and 6 at 1.7 M NaCl concentration;

FIG. 4: shows preserved platelets (%) for PVC tubing coated with dextran sulfates 2 and 3 at 0.25 M NaCl concentration;

FIG. 5: shows F1+2 (prothrombin fragment) for PVC tubing coated with dextran sulfates 2 and 3 at 0.25 M NaCl concentration;

FIG. 6: shows preserved platelets (%) for PVC tubing coated with dextran sulfates 4 and 5 at 0.25 M NaCl concentration;

FIG. 7: shows F1+2 (prothrombin fragment) for PVC tubing coated with dextran sulfates 4 and 5 at 0.25 M NaCl concentration;

FIG. 8: shows the coating thickness for PVC tubing coated with dextran sulfates 2, 4 and 6 at 0.25 M and 1.7 M NaCl concentration;

FIG. 9: shows heparin concentration (HC) for PVC tubing coated with dextran sulfates 1, 2, 4 and 6 at 1.7 M NaCl concentration;

FIG. 10: shows preserved platelets (%) for PVC tubing coated with dextran sulfates 4 and 6 at 1.7 M NaCl concentration, pre- and post-temperature and humidity test;

FIG. 11: shows F1+2 (prothrombin fragment) for PVC tubing coated with dextran sulfates 4 and 6 at 1.7 M NaCl concentration, pre- and post-temperature and humidity test.

### DETAILED DESCRIPTION OF THE INVENTION

### Solid Object

Any solid object can potentially be coated using the process of the invention, although such coatings and processes are particularly useful for medical devices, analytical devices, separation devices, and other industrial articles including membranes.

Solid objects may have a thromboresistant surface. In certain embodiments of the invention, the thromboresistant surface may exhibit a direct pharmacologic inhibition of the coagulation response by immobilization of an anticoagulant entity. In certain embodiments of the invention, the thromboresistant surface does not cause any appreciable clinically-significant adverse reactions such as thrombosis, haemolysis, platelet, leukocyte, and complement activation, and/or other blood-associated adverse event when in contact with blood.

In the art, the terms “hemocompatible”, “non-thrombogenic”, “anti-thrombogenic” and the like can typically be interpreted as being equivalent to the term “thromboresistant”.

In one embodiment, the solid object is a medical device. When the solid object is a medical device, it is suitably a thromboresistant medical device. Thus, in one embodiment the solid object is a thromboresistant medical device. For the purposes of this patent application, the term “medical device” refers to intracorporeal or extra-corporeal devices but more usually to intracorporeal medical devices.

Intracorporeal medical devices are devices which are used within the anatomy e.g. within the vasculature or other body lumen, space or cavity, typically to provide a therapeutic effect. Intracorporeal devices may be of long-term or temporary use. Devices of long-term use are left, in part or in whole, in the anatomy after the immediate surgical procedure to deliver them e.g. stents or stent-grafts. Devices for temporary or short-term use include those which are transiently inserted into a treatment region (i.e. inserted and then removed in the same surgical procedure), such as a medical balloon. In one embodiment, the solid object is an intracorporeal medical device.

Examples of intracorporeal medical devices which can be permanent or temporary intracorporeal medical devices include stents including bifurcated stents, balloon-expandable stents, self-expanding stents, neurovascular stents and flow diverting stents, stent-grafts including bifurcated stent-grafts, grafts including vascular grafts and bifurcated grafts, sheaths including retractable sheaths such as interventional diagnostic and therapeutic sheaths, large and standard bore endovascular delivery sheaths, arterial introducer sheaths with and without hemostatic control and with or without steering, micro-introducer sheaths, dialysis access sheaths, guiding sheaths, and percutaneous sheaths, dilators, occluders such as vascular occluders, embolic filters, embolectomy devices, catheters, artificial blood vessels, blood indwelling monitoring devices, valves including artificial heart valves, pacemaker electrodes, guidewires, cardiac leads, cardiopulmonary bypass circuits, cannulae, plugs, drug delivery devices, balloons, tissue patch devices, blood pumps, patches, lines such as chronic infusion lines or arterial lines, placement wires, devices for continuous subarachnoid infusions, feeding tubes, CNS shunts such as ventriculopleural shunts, ventriculoatrial (VA) shunts, ventriculoperitoneal (VP) shunts, ventricular atrial shunts, portosystemic shunts and shunts for ascites.

Examples of catheters include, but are not limited to, microcatheters, central venous catheters, peripheral intravenous catheters, hemodialysis catheters, catheters such as coated catheters include implantable venous catheters, tunnelled venous catheters, coronary catheters useful for angiography, angioplasty, or ultrasound procedures in the heart or in peripheral veins and arteries, catheters containing spectroscopic or imaging capabilities, hepatic artery infusion catheters, CVC (central venous catheters), peripheral intravenous catheters, peripherally inserted central venous catheters (PIC lines), flow-directed balloon-tipped pulmonary artery catheters, total parenteral nutrition catheters, chronic dwelling catheters (e.g. chronic dwelling gastrointestinal catheters and chronic dwelling genitourinary catheters), peritoneal dialysis catheters, CPB catheters (cardiopulmonary bypass), urinary catheters and microcatheters (e.g. for intracranial application).

In one embodiment, the solid object is an intracorporeal medical device selected from the group consisting of stents, stent-grafts, sheaths, dilators, occluders, valves, embolic filters, embolectomy devices, catheters, artificial blood vessels, blood indwelling monitoring devices, valves, pacemaker electrodes, guidewires, cardiac leads, cardiopulmonary bypass circuits, cannulae, plugs, drug delivery devices, balloons, tissue patch devices, blood pumps, patches, lines, placement wires, devices for continuous subarachnoid infusions, feeding tubes and shunts. In a specific embodiment, the solid object is a stent or a stent-graft.

In one embodiment, said intracorporeal medical device can be used in neurological, peripheral, cardiac, orthopaedic, dermal, or gynaecologic applications. In one embodiment, said stents can be used in cardiac, peripheral or neurological applications. In one embodiment, said stent-grafts can be used in cardiac, peripheral or neurological applications. In one embodiment, said sheaths can be used in carotid, renal, transradial, transseptal, paediatric or micro applications.

Examples of extracorporeal medical devices are blood treatment devices, and transfusion devices. In one embodiment, said intracorporeal medical device can be used in neurological, peripheral, cardiac, orthopaedic, dermal, or gynaecologic applications. In one embodiment the extracorporeal medical device is an oxygenator. In another embodiment the extracorporeal medical device is a filter capable of removing viruses, bacteria, sepsis-causing pro-inflammatory cytokines and toxins.

A membrane can be, for example, a haemodialysis membrane.

An analytical device can be, for example, a solid support for carrying out an analytical process such as chromatography or an immunological assay, reactive chemistry or catalysis. Examples of such devices include slides, beads, well plates and membranes.

A separation device can be, for example, a solid support for carrying out a separation process such as protein purification, affinity chromatography or ion exchange. Examples of such devices include filters and columns.

The solid object may comprise or be formed of a metal, a synthetic or naturally occurring organic or inorganic polymer, a ceramic material, a protein-based material, or a polysaccharide-based material, inter alia.

Suitable metals include, but are not limited to, biocompatible metals such as titanium, stainless steel, high nitrogen stainless steel, cobalt, chromium, nickel, tantalum, niobium, gold, silver, rhodium, zinc, platinum, rubidium, copper and magnesium, and combinations (alloys) thereof. Suitable alloys include cobalt-chromium alloys such as L-605, MP35N, Elgiloy, titanium alloys including nickel-titanium alloys (such as Nitinol), tantalum alloys, niobium alloys (e.g. Nb-1% Zr), and others.

In one embodiment, said biocompatible metal is a nickel-titanium alloy, such as Nitinol.

Synthetic or naturally occurring organic or inorganic polymers include polyolefins, polyesters (e.g. polyethylene terephthalate and polybutylene terephthalate), polyester ethers, polyester elastomer copolymers (e.g. such as those available from DuPont in Wilmington, Del. under the tradename of HYTREL®), fluorine-containing polymers, chlorine-containing polymers (e.g. polyvinyl chloride (PVC)), block copolymer elastomers (e.g. such as those copolymers having styrene end blocks, and midblocks formed from butadiene, isoprene, ethylene/butylene, ethylene/propene), block copolymers (e.g. styrenic block copolymers such as acrylonitrile-styrene and acrylonitrile-butadiene-styrene block copolymers, or block copolymers wherein the particular block copolymer thermoplastic elastomers in which the block copolymer is made up of hard segments of a polyester or polyamide and soft segments of polyether), polyurethanes, polyamides (e.g. nylon 12, nylon 11, nylon 9, nylon 6/9 and nylon 6/6), polyether block amides (e.g. PEBAX®), polyetheresteramide, polyimides, polycarbonates, polyphenylene sulfides, polyphenylene oxides, polyethers, silicones, polycarbonates, polyhydroxyethylmethacrylate, polyvinyl pyrrolidone, polyvinyl alcohol, rubber, silicone rubber, polyhydroxyacids, polyallylamine, polyallylalcohol, polyacrylamide, polyacrylic acid, polystyrenes, polytetrafluoroethylene, poly(methyl)methacrylates, polyacrylonitriles, poly(vinylacetates), poly(vinyl alcohols), polyoxymethylenes, polycarbonates, phenolics, amino-epoxy resins, cellulose-based plastics, and rubber-like plastics, bioresorbables (e.g. poly(D,L-lactide) and polyglycolids, and copolymers thereof and copolymers thereof), derivatives thereof and mixtures thereof. Combinations of these materials can be employed with and without cross-linking. Some of these classes are available both as thermosets and as thermoplastic polymers. As used herein, the term “copolymer” shall be used to refer to any polymer formed from two or more monomers, e.g. 2, 3, 4, 5 and so on and so forth.

Fluorinated polymers (fluorine-containing polymers) include fluoropolymers such as expanded polytetrafluoroethylene (ePTFE), polytetrafluoroethylene (PTFE), fluorinated ethylene-propylene (FEP), perfluorocarbon copolymers (such as tetrafluoroethylene perfluoroalkylvinyl ether (TFE/PAVE) copolymers and copolymers of tetrafluoroethylene (TFE) and perfluoromethyl vinyl ether (PMVE)), and combinations of the above with and without crosslinking between the polymer chains.

In one embodiment, the solid object comprises a polyether-block-amide, such as PEBAX®. In another embodiment, the solid object comprises a chlorine-containing polymer (e.g. PVC) or a fluorine-containing polymer (e.g. ePTFE).

Polymeric substrates may optionally be blended with fillers and/or colorants. Thus, suitable substrates include pigmented materials such as pigmented polymeric materials.

Ceramic substrates may include, but are not limited to, silicone oxides, aluminium oxides, alumina, silica, hydroxyapapitites, glasses, calcium oxides, polysilanols, and phosphorous oxide.

Protein-based materials include silk and wool. Polysaccharide-based materials include agarose and alginate.

### Anticoagulant Entity

An anticoagulant entity is an entity capable of interacting with mammalian blood to prevent or alleviate coagulation or thrombus formation.

Anticoagulant entities include heparin moieties, dermatan sulfate moieties, dermatan disulfate moieties, hirudin, eptifibatide, tirofibran, urokinase, D-Phe-Pro-Arg chloromethylketone, an RGD peptide-containing compound, AZX100 (a cell peptide that mimics HSP20, Capstone Therapeutics Corp., USA), platelet receptor antagonists, anti-thrombin antibodies, anti-platelet receptor antibodies, aspirin, prostaglandin inhibitors, platelet inhibitors (e.g. clopidogrel nitric oxide (NO), prostaglandines and abciximab), antiplatelet peptides, coumadins (i.e. vitamin K antagonists of the 4-hydroxycoumarin class e.g. warfarin), argatroban, thrombomodulin, anticoagulant proteins, anticoagulant enzymes (e.g. apyrase). In one embodiment, the anticoagulant entity is selected from the group consisting of heparin moieties, dermatan sulfate moieties and dermatan disulfate moieties.

In one embodiment, the anticoagulant entity is a glycosaminoglycan. In one embodiment, the anticoagulant entity is a thrombin inhibitor.

The term “heparin moiety” refers to a heparin molecule, a fragment of a heparin molecule, a derivative of a heparin molecule or an analogue of a heparin molecule.

In one embodiment, the anticoagulant entity is a heparin moiety. Suitably the heparin moiety is selected from the group consisting of full length heparin (native heparin), an alkali metal or alkaline earth metal salt of heparin (e.g. sodium heparin (e.g. Hepsal or Pularin), potassium heparin (e.g. Clarin), lithium heparin, calcium heparin (e.g. Calciparine) or magnesium heparin (e.g. Cutheparine)), a low molecular weight heparin (e.g. ardeparin sodium, tinzaparin or dalteparin), heparan sulfate, a heparinoid, a heparin-based compound, heparin having a hydrophobic counter-ion, a synthetic heparin composition capable of antithrombin-mediated inhibition of factor Xa (e.g. a “fondaparinux” composition (e.g. Arixtra from GlaxoSmithKline)) and a synthetic heparin derivative comprising at least the active pentasaccharide sequence from heparin (see for example Petitou et al., Biochimie, 2003, 85 (1-2):83-9). Additional heparin moieties include heparin modified by means of e.g. mild nitrous acid degradation (U.S. Pat. No. 4,613,665A, incorporated herein by reference) or periodate oxidation (U.S. Pat. No. 6,653,457B1, incorporated herein by reference) and other modification reactions known in the art where the activity of the heparin moiety is preserved. Heparin moieties also include such moieties bound to a linker or spacer as described below. In one embodiment, the heparin moiety is full length heparin.

Low molecular weight heparins may be prepared by, for example, oxidative depolymerisation, enzymatic degradation or deaminative cleavage.

U.S. Pat. No. 6,461,665B1 (Scholander; incorporated herein by reference) discloses improving the anti-thrombogenic activity of surface-immobilized heparin by treating the heparin prior to immobilization. The improvement is achieved by treating the heparin at elevated temperature or at elevated pH, or by contacting the heparin with nucleophilic catalysts such as amines, alcohols, thiols or immobilized amino, hydroxyl or thiol groups.

The anticoagulant entity is covalently immobilized on the surface of the solid object, therefore does not substantially elute or leach from the solid object. As discussed below, the anticoagulant entity can be covalently immobilized by various methods.

The anticoagulant entity is covalently attached to the outermost layer of cationic polymer.

The anticoagulant entity is suitably end-point attached to the cationic polymer, particularly when the anticoagulant entity is a heparin moiety. Thus, in one embodiment, the anticoagulant entity is an end-point attached anticoagulant moiety. In a particular embodiment, the anticoagulant entity is an end-point attached heparin moiety. Where applicable, the anticoagulant entity is preferably connected through its reducing end. Thus, in one embodiment, the anticoagulant entity is connected through its reducing end. In a particular embodiment, the anticoagulant entity is an end-point attached heparin moiety connected through its reducing end (sometimes referred to as position C1 of the reducing terminal). The advantage of end-point attachment, especially reducing end-point attachment, is that the biological activity of the anticoagulant entity (for example the heparin moiety) is maximized due to enhanced availability e.g. the antithrombin interaction sites as compared with attachment elsewhere in the anticoagulant entity (e.g. heparin moiety).

A representative end-point attachment process is described in EP0086186B1 (Larm; incorporated herein by reference in its entirety) which discloses a process for the covalent binding of oligomeric or polymeric organic substances to substrates of different types containing primary amino groups. The substance to be coupled, which may be heparin, is subjected to degradation by diazotization to form a substance fragment having a free terminal aldehyde group. The substance fragment is then reacted through its aldehyde group with the amino group of the substrate to form a Schiff's base, which is then converted (via reduction) to a secondary amine. The advantage of end-point attachment of heparin, especially reducing end point attachment (as described above in EP0086186B1), is that the biological activity of the heparin moiety is maximized due to enhanced availability of the antithrombin interaction sites as compared with attachment elsewhere in heparin moiety.

The anticoagulant entity may be covalently attached to the outermost layer of cationic polymer via a linker. Thus, in one embodiment, the anticoagulant entity is covalently attached via a linker.

In one embodiment, the linker comprises a secondary amine. A representative procedure for covalently bonding a heparin moiety to a polymer via a secondary amine is described in EP0086186B1.

In one embodiment, the linker comprises a secondary amide. A representative procedure for covalently bonding a heparin moiety to a polymer via an amidation reaction involving N-succinimidyl 3-(2-pyridyldithio)propionate (SPDP) or 1-ethyl-3-(3-dimethylaminopropyl)-carbodiimide (EDC) is set out in WO2012/123384A1 (incorporated herein by reference in its entirety).

In one embodiment, the linker comprises a 1,2,3-triazole. A representative procedure for covalently bonding a heparin moiety to a polymer via a 1,2,3-triazole linkage is described in WO2010/029189A2 (Carmeda AB, incorporated herein by reference in its entirety). The document describes the azide- or alkyne-functionalization of a polyimine; the preparation of alkyne- and azide-functionalized heparin (both native and nitrous acid degraded heparin); and reactions to link the derivatised heparin to the derivatised polymer via a 1,2,3-triazole linker.

In one embodiment, the linker comprises a thioether. A representative procedure for covalently bonding a heparin moiety to a polymer via a thioether linkage is described in WO2011/110684A1 (Carmeda AB et al., incorporated herein by reference in its entirety).

### Cationic Polymer

The cationic polymer may be a straight chain polymer but is more usually a branched chain polymer such as a hyperbranched polymer. In one embodiment the cationic polymer is a branched cationic polymer. The cationic polymer is optionally cross-linked. In one embodiment, the cationic polymer comprises primary/secondary amine groups. In one embodiment, the cationic polymer is a polyamine, which is optionally cross-linked. The cationic polymer (e.g. polyamine), suitably has molecular weight of 5 kDa-3,000 kDa, such as 5 kDa-2,000 kDa, 5 kDa-1,500 kDa, 5 kDa-1,000 kDa, 5 kDa-800 kDa, 5 kDa-500 kDa, 5 kDa-300 kDa or 5 kDa-200 kDa or 800 kDa-3,000 kDa. When the cationic polymer (e.g. polyamine) is cross-linked, it is suitably cross-linked using an aldehyde cross-linker such as crotonaldehyde and/or glutaraldehyde. In one embodiment, the cationic polymer is a polyalkyleneimine e.g. polyethyleneimine.

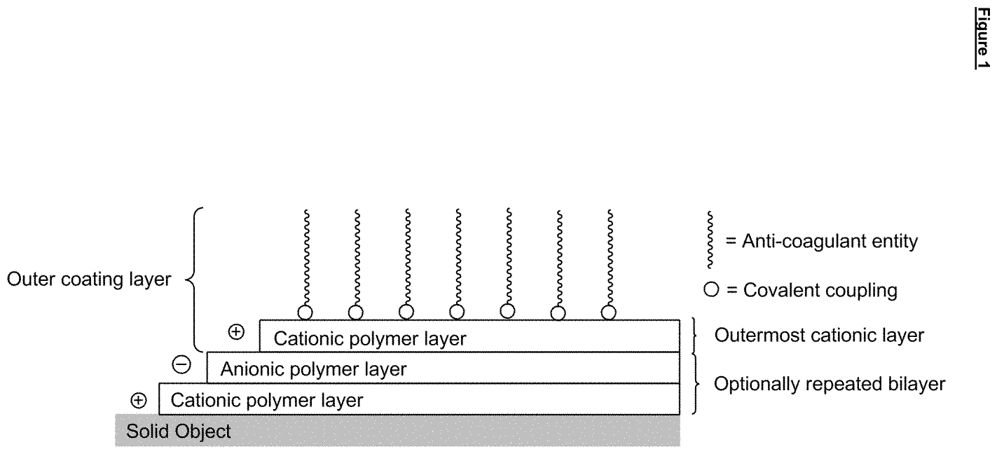

The cationic polymer forms part of a layer-by-layer coating of cationic polymer and anionic polymer, which is formed by alternately treating the surface of the solid object with layers of cationic and anionic polymer. A bilayer is defined herein as one layer of cationic polymer and anionic polymer. In the layer-by-layer coating, the cationic polymer is typically applied before the anionic polymer i.e. a surface of the solid object is typically first treated with a first layer of cationic polymer (step i) in claim 1), upon which a first layer of anionic polymer is applied (step ii) in claim 1). Depending on the number of bilayers required, further layers of cationic polymer and anionic polymer may be applied (step iii) in claim 1). When the final (which may be also the first) bilayer of cationic and anionic polymer is completed, a layer of cationic polymer is then applied (corresponding to step iv) in claim 1). This layer (i.e. the outermost layer) of cationic polymer is then treated with an anticoagulant entity, so as to covalently attach the anticoagulant entity to the layer of cationic polymer. Thus, the outer coating layer of cationic polymer can be said to “comprise” an anticoagulant entity. In the layer-by-layer coating, the innermost layer is a layer of cationic polymer and the outermost layer is an outer coating layer of cationic polymer to which the anticoagulant entity is covalently attached (see FIG. 1).

In one embodiment, the cationic polymer of step i) is a polyamine, which is optionally cross-linked. In one embodiment, the cationic polymer of step iv) is a polyamine, which is optionally cross-linked. In one embodiment, the cationic polymer of step i) is the same as the cationic polymer of step iv).

WO2012/123384A1 (Gore Enterprise Holdings, Inc. et al., incorporated herein by reference in its entirety) discloses a device with a coating comprising a plurality of hyperbranched polymer molecules bearing anticoagulant entities, in particular heparin. Such hyperbranched polymer molecules may be utilised in the outermost layer of cationic polymer i.e. such hyperbranched polymers may be used as the cationic polymer of step iv), and then modified to bear anticoagulant entities in step v).

### Anionic Polymer

Anionic polymers suitable for the invention carry deprotonated functional groups from the groups consisting of —COOH, —SO₃H and —PO₃H₂. Thus, in one embodiment, the anionic polymer is a polymer comprising groups selected from —CO₂⁻, —SO₃⁻, —PO₃H⁻ and —PO₃²⁻. Suitably the anionic polymer is a polymer comprising —SO₃⁻ groups. More suitably the anionic polymer is a polymer consisting of —SO₃⁻ groups.

The anionic polymer is suitably an anionic glycosaminoglycan or polysaccharide. The anionic characteristics of the polymer typically derive from carboxylate or sulfate groups along the polymer chain. Thus, in one embodiment, the anionic polymer is a glycosaminoglycan or polysaccharide bearing carboxylate and/or sulfate groups, in particular a glycosaminoglycan bearing carboxylate and/or sulfate groups. The anionic polymer may be branched or unbranched. In one embodiment, the anionic polymer is not heparin. In one embodiment, the anionic polymer and the anticoagulant entity are not the same.

In one embodiment, the anionic polymer is optionally cross-linked.

In one embodiment, the anionic polymer is selected from the group consisting of dextran sulfate, hyaluronic acid, poly(2-acrylamido-2-methyl-1-propanesulfonic acid), poly(2-acrylamido-2-methyl-1-propanesulfonic acid-co-acrylonitrile), acrylonitrile, poly(acrylic acid), polyanetholesulfonic acid, poly(sodium 4-styrenesulfonate), poly(4-styrenesulfonic acid-co-maleic acid), poly(vinyl sulfate), polyvinylsulfonic acid and salts thereof. Suitably, the anionic polymer is dextran sulfate.

Dextran sulfate is a sulfated polymer of anhydroglucose. The degree of sulfation and consequently the sulfur content of the dextran sulfate can vary.

In some embodiments the sulfur content is between 10% and 25% by weight, e.g. the sulfur content is between 15% and 20% by weight.

The anionic polymer is characterized by having a total molecular weight of 20 kDa-650 kDa. In one embodiment, the anionic polymer is characterized by having a total molecular weight of 20 kDa-125 kDa, such as 30 kDa-110 kDa. In one embodiment, the anionic polymer is characterized by having a total molecular weight of 20 kDa-75 kDa, such as 25 kDa-60 kDa. In one embodiment, the anionic polymer is characterized by having a total molecular weight of 75 kDa-125 kDa, such as 80 kDa-120 kDa. In one embodiment, the anionic polymer is characterized by having a total molecular weight of 525 kDa-650 kDa, such as 550 kDa-625 kDa. Suitably, the total molecular weight of the anionic polymer is measured according to Evaluation Method G.

The anionic polymer is characterized by having a solution charge density of ≤4 μeq/g. In one embodiment, the anionic polymer is characterized by having a solution charge density of between 1.5 μeq/g and ≤4 μeq/g, such as between 2 μeq/g and ≤4 μeq/g. Suitably, the solution charge density of the anionic polymer is measured according to Evaluation Method H.

The present inventors have found that, surprisingly, the charge density of the anionic polymer used in the layer-by-layer coating has a significant impact on the resulting characteristics, in particular the thromboresistant properties of the final solid objects of the invention. As shown in Example 2, the present inventors have found that solid objects with coatings comprising dextran sulfates with charge density of ≤4 μeq/g exhibited significantly higher preserved platelets and lower F1+2 values (i.e. properties of greater thromboresistance) compared with solid objects coated using comparable dextran sulfates with charge density of >4 μeq/g.

### Coating Bilayer(s) of Cationic and Anionic Polymer

The solid object of the invention has a surface comprising a layered coating of cationic and anionic polymer. As explained above, a bilayer is defined herein as one layer of cationic and anionic polymer (see FIG. 1).

The layered coating comprises one or more coating bilayers, e.g. 2 or more, 3 or more, 4 or more, 5 or more, 6 or more, 7 or more, 8 or more, 9 or more or 10 or more coating bilayers. When more than one coating bilayer is applied, steps i) and ii) are repeated i.e. step iii) is not optional. In one embodiment of the process of the invention, step iii) is not optional. In this embodiment, step iii) is repeated as many times as is necessary to achieve the required number of coating bilayers e.g. 1 time, 2 times, 3 times, 4 times, 5 times, 6 times, 7 times, 8 times or 9 times. In one embodiment of the process of the invention, in step iii), steps i) and ii) are repeated between 1 and 10 times, such 1, 2, 3, 4, 5 or 6 times.

When step iii) is not optional (i.e. when steps i) and ii) are repeated one or more times) the precise process conditions of each repeat need not be identical (e.g. the salt type and/or concentration used in treating the surface with an anionic polymer in step ii) need not be identical in each repetition). In an embodiment, the process conditions (e.g. the salt type and/or concentration used in treating the surface with an anionic polymer in step ii)) are identical in each repetition.

### Process Steps

In one aspect, the present invention provides a process for the manufacture of a solid object having a surface comprising a layered coating of cationic and anionic polymer, wherein the outer coating layer comprises cationic polymer to which is covalently bound an anticoagulant entity, comprising the steps of:

i) treating a surface of the solid object with a cationic polymer;

ii) treating the surface with an anionic polymer;

iii) optionally repeating steps i) and ii) one or more times;

iv) treating the surface with a cationic polymer; and

v) treating the outermost layer of cationic polymer with an anticoagulant entity, thereby to covalently attach the anticoagulant entity to the outermost layer of cationic polymer;

wherein, the anionic polymer is characterized by having (a) a total molecular weight of 20 kDa-650 kDa; and (b) a solution charge density of ≤4 μeq/g.

In another aspect is provided a solid object as described herein, obtainable by a process comprising the steps of:

i) treating a surface of the solid object with a cationic polymer;

ii) treating the surface with an anionic polymer;

iii) optionally repeating steps i) and ii) one or more times;

iv) treating the surface with a cationic polymer; and

v) treating the outermost layer of cationic polymer with an anticoagulant entity, thereby to covalently attach the anticoagulant entity to the outermost layer of cationic polymer;

wherein, the anionic polymer is characterized by having (a) a total molecular weight of 20 kDa-650 kDa; and (b) a solution charge density of ≤4 μeq/g.

It should be noted that steps i)-v) are carried out sequentially in the given order i.e. each of steps i)-iv) is implicitly followed by “and then”. This does not preclude one or more additional steps being carried out between each of specified steps i)-v). Thus, in one embodiment, the process of the invention additionally comprises a step between step i) and step ii), between step ii) and step iii), between step iii) and step iv) or between step iv) and step v).

It should be understood, for example, that washing steps may be performed between the specified process steps.

In one embodiment, the anionic polymer is applied to the surface at a salt concentration of 0.05 M-3.0 M, such as 0.05 M-2.0 M, 0.05 M-1.5 M, 0.05 M-1.0 M, 0.1 M-1.0 M or 0.2 M-1.0 M.

In one embodiment, step ii) is carried out at a salt concentration of 0.05 M-3.0 M, such as 0.05 M-2.0 M, 0.05 M-1.5 M, 0.05 M-1.0 M, 0.1 M-1.0 M or 0.2 M-1.0 M.

In one embodiment, the salt is an inorganic salt. Suitably, the salt is selected from the group consisting of a sodium salt, a potassium salt, a magnesium salt, a calcium salt, a lithium salt, an ammonium salt, a barium salt and a strontium salt.

In one embodiment, the salt is an inorganic sodium salt.

In one embodiment, the salt is selected from the group consisting of sodium chloride, sodium sulfate, sodium hydrogen phosphate and sodium phosphate.

In one embodiment, the salt is sodium chloride.

In one embodiment, the salt is not sodium chloride.

In one embodiment, the salt is sodium chloride at a concentration of 0.05 M-3.0 M e.g. 0.05 M-2.0 M.

In one embodiment, the salt is sodium sulfate at a concentration of 0.05 M-1.5 M e.g. 0.05 M-1.0 M.

In one embodiment, the salt is sodium hydrogen phosphate at a concentration of 0.05 M-3.0 M e.g. 0.05 M-2.0 M.

In one embodiment, the salt is sodium phosphate at a concentration of 0.05 M-3.0 M e.g. 0.05 M-2.0 M.

Prior to step i) (treating the surface of the solid object with a cationic polymer) the surface of the solid object can optionally be subjected to a pre-treatment step.

The pre-treatment step may be a cleaning step to improve adhesion and surface coverage of the subsequent coating. Suitable cleaning agents include solvents such as alcohols, solutions with high pH like solutions comprising a mixture of an alcohol and an aqueous solution of a hydroxide compound (e.g. sodium hydroxide), sodium hydroxide solution as such, solutions containing tetramethyl ammonium hydroxide (TMAH), acidic solutions like Piranha (a mixture of sulfuric acid and hydrogen peroxide), basic Piranha solution, and other oxidizing agents including combinations of sulfuric acid and potassium permanganate or different types of peroxysulfuric acid or peroxydisulfuric acid solutions (also as ammonium, sodium, and potassium salts), or by subjecting the solid object to plasma in air, argon or nitrogen atmosphere or combinations thereof.

Thus, in one embodiment, the process of the invention additionally comprises a pre-treatment step before step i). Suitably, the pre-treatment step is a cleaning step.

Alternatively, a pre-treatment step may involve overlaying the surface of the solid object to be coated according to steps i)-v) with a material such as a polymer or primer coating layer, prior to the application of steps i)-v). This “preparative” coating layer could, for example, allow the surface of solid object to be coated to be “sculpted” or modified to create a desired surface topography or texture in order to optimize the subsequent layered coating process. The additional coating layer could also improve the adherence of the subsequent layered coating, in particular helping to maintain its integrity during processing. An example of such a priming coating layer on a solid object is a coating layer applied using chemical vapour deposition (CVD). Another example of such a priming coating layer on a solid object is a coating of polydopamine or an analogue thereof.

In one embodiment, the pre-treatment step comprises treating a surface of the solid object with a polymer selected from the group consisting of a polyolefin, polyisobutylene, ethylene-α-olefin copolymers, an acrylic polymer, an acrylic copolymer, polyvinyl chloride, polyvinyl methyl ether, polyvinylidene fluoride, polyvinylidene chloride, a fluoropolymer (e.g. expanded polytetrafluoroethylene (ePTFE), polytetrafluoroethylene (PTFE), fluorinated ethylene-propylene (FEP), perfluorocarbon copolymers, e.g. tetrafluoroethylene perfluoroalkylvinyl ether (TFE/PAVE) copolymers, copolymers of tetrafluoroethylene (TFE) and perfluoromethyl vinyl ether (PMVE), copolymers of TFE with functional monomers that comprise acetate, alcohol, amine, amide, sulfonate, functional groups and the like as described in U.S. Pat. No. 8,658,707 (W.L. Gore and Associates, incorporated herein by reference in its entirety, as well as combinations thereof), polyacrylonitrile, a polyvinyl ketone, polystyrene, polyvinyl acetate, an ethylene-methyl methacrylate copolymer, an acrylonitrile-styrene copolymer, an ABS resin, Nylon 12, a block copolymer of Nylon 12, polycaprolactone, a polyoxymethylene, a polyether, an epoxy resin, a polyurethane, rayon-triacetate, cellulose, cellulose acetate, cellulose butyrate, cellophane, cellulose nitrate, cellulose propionate, a cellulose ether, carboxymethyl cellulose, a chitin, polylactic acid, polyglycolic acid, a polylactic acid-polyethylene oxide copolymer, polyethylene glycol, polypropylene glycol, polyvinyl alcohol, an elastomeric polymer such as silicone (e.g. polysiloxane or a substituted polysiloxane), a polyurethane, a thermoplastic elastomer, an ethylene vinyl acetate copolymer, a polyolefin elastomer, an EPDM rubber, and mixtures thereof.

In one embodiment, is provided a process for the manufacture of a solid object as described herein, consisting of steps i)-v) as defined herein i.e. the solid object has no additional coating layers beyond those resulting from steps i)-v).

A solid object coated according to the process of the invention may be sterilized. Suitable sterilization processes include, but are not limited to, sterilization using ethylene oxide, vapour hydrogen peroxide, plasma phase hydrogen peroxide, dry heat, autoclave steam sterilization, chlorine dioxide sterilization, gamma ray sterilization or electron beam sterilization.

As shown in Example 6, solid objects of the invention subjected to increased temperature and humidity retained their thromboresistant properties. Conditions of increased temperature and humidity can act as a mimic for the rigorous conditions of sterilization, in particular ethylene oxide sterilization. Hence, a solid object coated according to the process of the invention is expected to be stable to sterilization.

In one aspect of the invention is provided a solid object as described herein which has been sterilized, e.g. ethylene oxide sterilized.

### Coating Properties

Typically, the coating layer will have an average total thickness of ≤300 nm, e.g. ≤200 nm, ≤150 nm, ≤100 nm, ≤75 nm, ≤50 nm, ≤40 nm, ≤30 nm or ≤25 nm. In one embodiment, the solid object has coating thickness of about 10 nm to about 300 nm, e.g. about 10 nm to about 250 nm about 10 nm to about 200 nm, about 10 nm to about 150 nm, about 10 nm to about 100 nm, about 10 nm to about 75 nm, about 10 nm to about 50 nm, about 10 nm to about 40 nm, about 10 nm to about 30 nm or about 10 nm to about 25 nm. In one embodiment, the solid object has coating thickness of about 20 nm to about 300 nm, e.g. about 20 nm to about 200 nm, about 20 nm to about 150 nm, about 20 nm to about 100 nm, about 20 nm to about 75 nm, about 20 nm to about 50 nm, about 20 nm to about 40 nm, about 20 nm to about 30 nm or about 20 nm to about 25 nm. Coating thickness can be measured using a suitable coating thickness analyser or gauge, by using X-ray photoelectron spectroscopy with depth profiling (see Evaluation Method J), or by using Quartz Crystal Microbalance with Dissipation (see Evaluation Method N). Suitably the coating thickness is measured using Evaluation Method N.

As shown in Example 3, the present inventors have found that the effect of the molecular weight of the dextran sulfate used in the layer-by-layer coating process impacts on the overall coating thickness, and that depending on the charge density of the dextran sulfate used, the salt concentration used when applying the dextran sulfate layer(s) can be varied to further modify the resulting coating thickness.

In one embodiment, the solid object coated according to the process of the invention has anticoagulant entity activity (in particular heparin activity) of at least 1 pmol/cm² of surface e.g. at least 2 pmol/cm² of surface, at least 3 pmol/cm² of surface, at least 4 pmol/cm² of surface, or at least 5 pmol/cm² of surface for binding of ATIII, suitably measured according to Evaluation Method B.

In one embodiment, a thromboresistant surface of the solid object has anticoagulant entity activity (in particular heparin activity) of at least 1 pmol/cm² of surface e.g. at least 2 pmol/cm² of surface, at least 3 pmol/cm² of surface, at least 4 pmol/cm² of surface, or at least 5 pmol/cm² of surface for binding of ATIII, suitably measured according to Evaluation Method B.

In one embodiment, the solid object coated according to the process of the invention has anticoagulant entity activity (in particular heparin activity) of at least 5 pmol/cm² of surface e.g. at least 12 pmol/cm² of surface, at least 20 pmol/cm² of surface, at least 50 pmol/cm² of surface for binding of HCII, suitably measured according to Evaluation Method M.

In one embodiment, a thromboresistant surface of the solid object has anticoagulant entity activity (in particular heparin activity) of at least 5 pmol/cm² of surface e.g. at least 12 pmol/cm² of surface, at least 20 pmol/cm² of surface, at least 50 pmol/cm² of surface for binding of HCII, suitably measured according to Evaluation Method M.

In one embodiment, the solid object coated according to the process of the invention has blood contact performance of at least 80% preserved platelets, e.g. at least 85% preserved platelets, e.g. at least 90% preserved platelets, suitably measured according to Evaluation Method E.

In one embodiment, a thromboresistant surface of a solid object has blood contact performance of at least 80% preserved platelets, e.g. at least 85% preserved platelets, e.g. at least 90% preserved platelets, suitably measured according to Evaluation Method E.

In one embodiment, the solid object coated according to the process of the invention has an F1+2 value of <10,000 pmol/L e.g. less than 7,500 pmol/L, less than 5,000 pmol/L or less than 4,000 pmol/L, suitably measured according to Evaluation Method F.

In one embodiment, a thromboresistant surface of a solid object has an F1+2 value of <10,000 pmol/L, e.g. less than 7,500 pmol/L, less than 5,000 pmol/L or less than 4,000 pmol/L, suitably measured according to Evaluation Method F.

In one embodiment, the anticoagulant entity is a heparin moiety, and wherein the solid object has heparin concentration of at least 1 μg/cm², e.g. at least 2 μg/cm², at least 4 μg/cm², at least 5 μg/cm², or at least 6 μg/cm², suitably measured according to Evaluation Method A (see Example 4).

### Therapeutic Methods

Solid objects of the invention (e.g. medical devices), in particular as coated according to the process of the invention as described hereinabove are of use in medical therapy.

In one aspect of the invention is provided a solid object (in particular a medical device) as described hereinabove for use in treating tissue in the human or animal body. In another aspect of the invention is provided a solid object (in particular a medical device) coated according to the process of the invention described hereinabove for use in treating tissue in the human or animal body. The tissue to be treated includes any body cavity, space, or hollow organ passage(s) such as blood vessels, the urinary tract, the intestinal tract, nasal cavity, neural sheath, intervertebral regions, bone cavities, oesophagus, intrauterine spaces, pancreatic and bile ducts, rectum, and those previously intervened body spaces that have implanted vascular grafts, stents, prosthesis, or other type of medical implants. In yet another aspect of the invention, a solid object (e.g. a medical device) according to the invention as described hereinabove may be deployed to treat aneurysms in the brain. In yet another aspect of the invention, a solid object (e.g. a medical device) coated according to a process of the invention as described hereinabove may be deployed to treat aneurysms in the brain.

The coated solid object (in particular medical device) as described herein can be of use in the removal of obstructions such as emboli and thrombi from blood vessels, as a dilation device to restore patency to an occluded body passage, as an occlusion device to selectively deliver a means to obstruct or fill a passage or space, and as a centering mechanism for transluminal instruments like catheters.

In one embodiment is provided a solid object (in particular a medical device such as a stent, graft or stent-graft) as described hereinabove for use in the prevention or treatment of stenosis or restenosis in a blood vessel of the human body. In another embodiment is provided a solid object (in particular a medical device such as a stent, graft or stent-graft) as described hereinabove for use in the prevention or treatment of stenosis or restenosis in a blood vessel of the human body, where previously placed eluting constructs have failed. In another embodiment, a solid object (in particular a medical device such as a stent, graft or stent-graft) coated as described hereinabove can be used to establish or maintain arteriovenous access sites, e.g. those used during kidney dialysis. In a further embodiment, solid object (in particular a medical device such as a stent, graft or stent-graft e.g. a vascular graft) as described hereinabove may be used to redirect flow around an area of blockage or vessel narrowing. In another embodiment, a solid object (in particular a stent, graft or stent-graft) as described hereinabove may be deployed to restore patency to an area of diseased vessel or to exclude an aneurysm. In yet another embodiment, a solid object (in particular a medical device such as a stent, graft or stent-graft) as described hereinabove may be deployed to reinforce a diseased vessel following angioplasty. In yet another embodiment, a solid object (in particular a medical device such as a stent, graft or a stent-graft) as described hereinabove may be deployed in the brain using balloon assisted or coil assisted procedures.

In one embodiment is provided a solid object (in particular a medical device such as a stent, graft or stent-graft) coated according to the process of the invention as described hereinabove for use in the prevention or treatment of stenosis or restenosis in a blood vessel of the human body. In another embodiment is provided a solid object (in particular a medical device such as a stent, graft or stent-graft) coated according to the process of the invention as described hereinabove for use in the prevention or treatment of stenosis or restenosis in a blood vessel of the human body, where previously placed eluting constructs have failed. In another embodiment, a solid object (in particular a medical device such as a stent, graft or stent-graft) coated according to the process of the invention as described hereinabove can be used to establish or maintain arteriovenous access sites, e.g., those used during kidney dialysis. In a further embodiment, a solid object (in particular a medical device such as a stent, graft or stent-graft e.g. a vascular graft) coated according to the process of the invention described hereinabove may be used to redirect flow around an area of blockage or vessel narrowing. In another embodiment, solid object (in particular a medical device such as a stent, graft or stent-graft) coated according to the process of the invention as described hereinabove may be deployed to restore patency to an area of diseased vessel or to exclude an aneurysm. In yet another embodiment, a solid object (in particular a medical device such as a stent, graft or stent-graft) coated according to the process of the invention as described hereinabove may be deployed to reinforce a diseased vessel following angioplasty. In yet another embodiment, a solid object (in particular a medical device such as a stent, graft or a stent-graft) coated according to the process of the invention as described hereinabove may be deployed in the brain using balloon assisted or coil assisted procedures.

In one embodiment, a solid object (in particular a medical device) as described hereinabove can be used for Percutaneous Transluminal Angioplasty (PTA) in patients with obstructive disease of the peripheral arteries.

In one embodiment, a solid object (in particular a medical device) coated according to the process of the invention as described hereinabove can be used for Percutaneous Transluminal Angioplasty (PTA) in patients with obstructive disease of the peripheral arteries.

In one aspect of the invention is provided a method for the prevention or treatment of stenosis or restenosis which comprises implanting into said blood vessel in the human body a solid object (in particular a medical device) as described hereinabove. In another aspect of the invention is provided a method for the prevention or treatment of stenosis or restenosis which comprises implanting into said blood vessel in the human body a solid object (in particular a medical device) coated according to the process of the invention as described hereinabove.

### Further Embodiments of the Invention

Embodiments and preferences described above with respect to the solid object and process of the invention apply equally to embodiments below.

In one embodiment is provided a solid object having a surface comprising a layered coating of cationic and anionic polymer, wherein the outer coating layer is a layer comprising cationic polymer;

and wherein the anionic polymer is characterized by having (a) a total molecular weight of 20 kDa-650 kDa; and (b) a solution charge density of ≤4 μeq/g.

In one embodiment is provided a solid object obtainable by a process comprising the steps of:

i) treating a surface of the solid object with a cationic polymer;

ii) treating the surface with an anionic polymer;

iii) optionally repeating steps i) and ii) one or more times; and

iv) treating the surface with a cationic polymer;

wherein, the anionic polymer is characterized by having (a) a total molecular weight of 20 kDa-650 kDa; and (b) a solution charge density of ≤4 μeq/g.

In one embodiment is provided a process for the manufacture of a solid object having a surface comprising a layered coating of cationic and anionic polymer, wherein the outer coating layer comprises cationic polymer, comprising the steps of:

i) treating a surface of the solid object with a cationic polymer;

ii) treating the surface with an anionic polymer;

iii) optionally repeating steps i) and ii) one or more times; and

iv) treating the surface with a cationic polymer;

wherein, the anionic polymer is characterized by having (a) a total molecular weight of 20 kDa-650 kDa; and (b) a solution charge density of ≤4 μeq/g.

In one embodiment is provided a solid object having a surface comprising a layered coating of cationic and anionic polymer, wherein the outer coating layer is a layer comprising anionic polymer; and wherein the anionic polymer is characterized by having (a) a total molecular weight of 20 kDa-650 kDa; and (b) a solution charge density of ≤4 μeq/g.

In one embodiment is provided a solid object obtainable by a process comprising the steps of:

i) treating a surface of the solid object with a cationic polymer;

ii) treating the surface with an anionic polymer; and

iii) optionally repeating steps i) and ii) one or more times;

wherein, the anionic polymer is characterized by having (a) a total molecular weight of 20 kDa-650 kDa; and (b) a solution charge density of ≤4 μpeq/g.

In one embodiment is provided a process for the manufacture of a solid object having a surface comprising a layered coating of cationic and anionic polymer, wherein the outer coating layer comprises anionic polymer, comprising the steps of:

i) treating a surface of the solid object with a cationic polymer;

ii) treating the surface with an anionic polymer; and

iii) optionally repeating steps i) and ii) one or more times;

wherein, the anionic polymer is characterized by having (a) a total molecular weight of 20 kDa-650 kDa; and (b) a solution charge density of ≤4 μeq/g.

In one embodiment is provided a process for the manufacture of a solid object having a surface comprising a layered coating of cationic and anionic polymer, wherein the outer coating layer comprises cationic polymer to which is covalently bound an anticoagulant entity, consisting of the steps of:

i) treating a surface of the solid object with a cationic polymer;

ii) treating the surface with an anionic polymer;

iii) optionally repeating steps i) and ii) one or more times;

iv) treating the surface with a cationic polymer; and
v) treating the outermost layer of cationic polymer with an anticoagulant entity, thereby to covalently attach the anticoagulant entity to the outermost layer of cationic polymer;

wherein, the anionic polymer is characterized by having (a) a total molecular weight of 20 kDa-650 kDa; and (b) a solution charge density of ≤4 μeq/g.

In one embodiment is provided a solid object as described herein, obtainable by a process consisting of the steps of:

i) treating a surface of the solid object with a cationic polymer;

ii) treating the surface with an anionic polymer;

iii) optionally repeating steps i) and ii) one or more times;

iv) treating the surface with a cationic polymer; and

v) treating the outermost layer of cationic polymer with an anticoagulant entity, thereby to covalently attach the anticoagulant entity to the outermost layer of cationic polymer;

wherein, the anionic polymer is characterized by having (a) a total molecular weight of 20 kDa-650 kDa; and (b) a solution charge density of ≤4 μeq/g.

### Additional Clauses of the Invention

1. A solid object having a surface comprising a layered coating of cationic and anionic polymer, wherein the outer coating layer is a layer comprising cationic polymer to which is covalently bound an anticoagulant entity;

and wherein the anionic polymer is characterized by having (a) a total molecular weight of 20 kDa-650 kDa; and (b) a solution charge density of ≤4 μeq/g.

2. A solid object according to clause 1 obtainable by a process comprising the steps of:
i) treating a surface of the solid object with a cationic polymer;
ii) treating the surface with an anionic polymer;
iii) optionally repeating steps i) and ii) one or more times;
iv) treating the surface with a cationic polymer; and
v) treating the outermost layer of cationic polymer with an anticoagulant entity, thereby to covalently attach the anticoagulant entity to the outermost layer of cationic polymer;
wherein, the anionic polymer is characterized by having (a) a total molecular weight of 20 kDa-650 kDa; and (b) a solution charge density of ≤4 μeq/g.

3. A process for the manufacture of a solid object having a surface comprising a layered coating of cationic and anionic polymer, wherein the outer coating layer comprises cationic polymer to which is covalently bound an anticoagulant entity, comprising the steps of:
i) treating a surface of the solid object with a cationic polymer;
ii) treating the surface with an anionic polymer;
iii) optionally repeating steps i) and ii) one or more times;
iv) treating the surface with a cationic polymer; and
v) treating the outermost layer of cationic polymer with an anticoagulant entity, thereby to covalently attach the anticoagulant entity to the outermost layer of cationic polymer;
wherein, the anionic polymer is characterized by having (a) a total molecular weight of 20 kDa-650 kDa; and (b) a solution charge density of ≤4 μeq/g.

4. A solid object according to clause 1 or clause 2, or a process for the manufacture of a solid object according to clause 3, wherein the anionic polymer is dextran sulfate.

5. A solid object or a process for the manufacture of a solid object according to any one of clauses 1 to 4, wherein the anionic polymer is characterized by having a total molecular weight of 20 kDa-125 kDa such as 20 kDa-75 kDa or 75 kDa-125 kDa.

6. A solid object or a process for the manufacture of a solid object according to any one of clauses 1 to 4, wherein the anionic polymer is characterized by having a total molecular weight of 525 kDa-650 kDa.

7. A solid object or a process for the manufacture of a solid object according to any one of clauses 1 to 6, wherein the anionic polymer is characterized by having a solution charge density of between 1.5 μeq/g and μeq/g, such as between 2 μeq/g and ≤4 μeq/g.

8. A solid object or a process for the manufacture of a solid object according to any one of clauses 1 to 7, wherein the anionic polymer is applied to the surface at a salt concentration of 0.05 M-3.0 M, such as 0.05 M-2.0 M, 0.05 M-1.5 M, 0.05 M-1.0 M, 0.1 M-1.0 M or 0.2 M-1.0 M.

9. A solid object according to clause 1 or any one of clauses 4 to 8, wherein the cationic polymer is a polyamine, which is optionally cross-linked.

10. A process for the manufacture of a solid object according to any one of clauses 3 to 7, wherein the cationic polymer of step i) and/or step iv) is a polyamine, which is optionally cross-linked.

11. A solid object or a process for the manufacture of a solid object according to any one of clauses 1 to 10, wherein the anticoagulant entity is a heparin moiety, e.g. an end-point attached heparin moiety which is connected through its reducing end.

12. A solid object or a process for the manufacture of a solid object according to any one of clauses 1 to 11, wherein the solid object is a thromboresistant medical device.

13. A solid object or a process for the manufacture of a solid object according to any one of clauses 1 to 12, with coating thickness of ≤300 nm e.g. ≤250 nm, ≤200 nm, ≤150 nm, ≤100 nm, ≤75 nm, ≤50 nm, ≤40 nm, ≤30 nm or ≤25 nm.

14. A solid object having a surface comprising a layered coating of cationic and anionic polymer, wherein the outer coating layer is a layer comprising cationic polymer; and wherein the anionic polymer is characterized by having (a) a total molecular weight of 20 kDa-650 kDa; and (b) a solution charge density of ≤4 μeq/g.

15. A solid object having a surface comprising a layered coating of cationic and anionic polymer, wherein the outer coating layer is a layer comprising anionic polymer; and wherein the anionic polymer is characterized by having (a) a total molecular weight of 20 kDa-650 kDa; and (b) a solution charge density of ≤4 μeq/g.

### Advantages

Solid objects coated according to the process of the invention, at least in some embodiments, are expected to have one or more of the following merits or advantages:
A coating of the anticoagulant entity having uniform distribution and being comparatively smooth can be obtained e.g. as determined using Evaluation Method C (toluidine blue staining test) or Evaluation Method I (SEM);
A uniform coating may be obtained which will mask the intrinsic properties of the solid object, for example to improve the thromboresistant properties of a device irrespective of the material of its manufacture;
A coating with good anticoagulant entity activity such as heparin activity can be obtained e.g. as determined using Evaluation Method B or M;
A thromboresistant coating which does not leach anticoagulant entity e.g. heparin, due to its covalent attachment and therefore has a long lifetime may be obtained;
A coating whose properties are preserved upon sterilization (e.g. with EO) may be obtained;
A self-healing coating may be obtained due to the possibility of reversible forming of ionic interactions between the layers;
A coating with good biocompatibility can be obtained e.g. as determined by using Evaluation Method D;
A coating which may reduce the need for systemic administration of anticoagulant e.g. heparin, and reduce the likelihood of contact activation e.g. as determined using Evaluation Method E (platelets) and/or Evaluation Method F (blood loop) may be obtained;
A solid object having a combination of anti-inflammatory properties as determined by using Evaluation Method D and thromboresistance can be obtained which may be beneficial in certain applications e.g. cardiovascular applications;
An analytical or separation device with good binding capacity to biomolecules may be obtained; and
An analytical or separation device with long heparin activity life time may be obtained.

The invention embraces all combinations of indicated groups and embodiments of groups recited above.

### ABBREVIATIONS

ABS acrylonitrile butadiene styrene

ATIII antithrombin III

CNS central nervous system

CPB cardiopulmonary bypass

CVC central venous catheter

CVD chemical vapour deposition

Da dalton

EDC 1-ethyl-3-(3-dimethylaminopropyl)-carbodiimide

EO ethylene oxide

EPDM ethylene propylene diene monomer (M-class)

ePTFE expanded polytetrafluoroethylene

FEP fluorinated ethylene-propylene

GPC gel permeation chromatography

HCII heparin cofactor II

HIT heparin induced thrombocytopenia

IEP isoelectric point

M molar concentration

MBTH 3-methyl-2-benzothiazolinone hydrazone hydrochloride

PAVE perfluoroalkylvinyl ether

PES-Na sodium polyethylene sulfate

PTA percutaneous transluminal angioplasty

PIC peripherally inserted central catheter

PMVE perfluoromethyl vinyl ether

PTFE polytetrafluoroethylene

PUR polyurethane

PVC polyvinyl chloride

QCM quartz crystal microbalance

RGD arginylglycylaspartic acid

SEM scanning electron microscopy/microscope

SPDP N-succinimidyl 3-(2-pyridyldithio)propionate

TFE tetrafluoroethylene

TMAH tetramethyl ammonium hydroxide

TMB 3,3′,5,5′-tetramethylbenzidine

VA ventriculoatrial

VP ventriculoperitoneal

XPS X-ray photoelectron spectroscopy

### EXAMPLES

### General Procedures

### Chemicals

Isopropanol, sodium dihydrogen phosphate dihydrate, sodium sulfate and sodium chloride are available from Sigma Aldrich and VWR Chemicals and may be used as received. Heparin of pharmacopea quality was treated with nitrous acid, essentially as described in EP0086186A1 and used in the Examples. Polyamines are available from vendors as described in U.S. Pat. No. 9,101,696B2. Dextran sulfates were purchased from various vendors as indicated in Table 1 of Example 1. DI water was used in the Examples below.

### Materials

PVC tubing was purchased from Flex Tubing Products. Quartz crystal microbalance crystals covered with gold were obtained from Q-sense (QSX 301).

### Evaluation Methods

The parameter being evaluated by each method is given in parentheses.

### Evaluation Method A: Heparin Concentration Test (Quantitative Heparin Attachment)

Quantification of surface immobilized heparin can be performed by complete degradation of heparin followed by colorimetric determination of the reaction products released into solution. Degradation is achieved by reacting the heparin surface with an excess of sodium nitrite under acidic conditions. The degradation products, mainly disaccharides, are quantified colorimetrically in a reaction with MBTH (3-methyl-2-benzothiazolinone hydrazone hydrochloride), essentially as described in Smith R. L. and Gilkerson E (1979), Anal Biochem 98, 478-480, which is incorporated herein by reference in its entirety.

### Evaluation Method B: Heparin Activity Test (Quantitative Heparin Function Using ATIII)

For solid objects of the invention comprising a heparin coating, the heparin activity of the device can be measured by measuring the ability, or capacity, of the heparin to bind antithrombin III (ATIII) as described by Pasche, et al. in “A binding of antithrombin to immobilized heparin under varying flow conditions” (Artif. Organs 1991; 15:281-491, incorporated herein by reference in its entirety) and Larsen M. L, et al. in “Assay of plasma heparin using thrombin and the chromogenic substrate H-D-Phe-Pip-Arg-pNA” (S-2238) (Thromb. Res. 1978; 13:285-288, incorporated herein by reference in its entirety), and can be used to evaluate a solid object's thromboresistant properties. Washed samples are incubated with an excess of antithrombin in solution to saturate all available antithrombin-binding sites of the heparin surface. Non-specifically adsorbed antithrombin is rinsed away using a salt solution. Subsequently, antithrombin specifically bound to the surface bound heparin is released by incubating with a solution of heparin at high concentration. Finally, the antithrombin released from the heparin surface is measured in a thrombin inhibition assay, based on a chromogenic thrombin substrate. The results are expressed as picomoles antithrombin III (ATIII) bound per apparent square centimeter of device (pmol ATIII/cm² solid object surface). The apparent solid object surface area does not take into account multiple covered surfaces nor porosity considerations of a solid object composed of a porous material. If the surface of the solid object is porous, the effect of porosity on surface area is not considered for these calculations. For example, the apparent surface area of a cylindrical tubular ePTFE vascular graft (which is made of a porous material) with heparin immobilized on substrate material comprising the inner surface of the tubular graft is calculated as it is for any cylindrical geometry as 2 πrL: where r is the graft inner radius; L is the axial length; and π is the number pi. This method can be used to measure the activity of any anticoagulant entity with ATIII binding activity.

### Evaluation Method C: Toluidine Blue Staining Test (Heparin Distribution)

Heparin distribution is evaluated using toluidine blue staining solution. The solution is prepared by dissolving 200 mg of toluidine blue in 1 L of water. The samples are subjected to the staining solution for 2 minutes prior to extensive water rinse. A blue/violet staining indicates that negatively charged heparin molecules are homogenously distributed in the outer coating layer.

### Evaluation Method D—Surface Biocompatibility

The biocompatibility of a surface of a solid object coated according to a process of the invention can be assessed as described in Lappegard, K. T 2008, J. Biomed. Mater. Res. Vol 87, 129-135 (incorporated herein by reference in its entirety). A procedure which may be used to evaluate the inflammatory response is as follows. Firstly, the coated solid object is washed with 0.15 M saline solution for 15 min. The wetted coated solid object is placed in heparinized PVC tubing containing whole blood and left to rotate in a circulating loop at 20 rpm (see Ekdahl K. N., Advances in Experimental Medicine and Biology, 2013, 735, 257-270 (incorporated herein by reference in its entirety) for a representative procedure). After incubation, the blood is centrifuged for 15 min, 3220 g at 4° C. The plasma is frozen in aliquots at −70 ° C. for later analysis of cytokines. Plasma samples are analyzed using multiplex cytokine assay (Bio-Plex Human Cytokine 27-Plex Panel, Bio-Rad Laboratories, Hercules, Calif.) according to the method described by Lappegard et al. (above).

The negative control is an empty loop of heparinized PVC without any device. This represents a non-inflammatory control for which the incubated blood should demonstrate no or minimal amount of inflammatory markers. The positive control is an empty loop of non-heparinized PVC without any device. This represents an inflammatory control for which a greater amount of inflammatory markers should be observed. The controls are included for ensuring the quality of the experiment and the blood.

### Evaluation Method E: Blood Loop Evaluation Test (Measurement of Platelet Loss)

Blood contact evaluation can be performed on a coated object to evaluate its thromboresistant properties. A procedure which may be used when the solid object is a tubular device such as a piece of PVC tubing is as follows. Firstly, the luminal side of the coated tubing is washed with 0.15 M saline solution for 15 hours at a flow of 1 mL/min to ensure complete wetting and removal of any loosely bound anticoagulant entity, such that a stable surface remains. The washed tubing is then incubated in a Chandler loop model performed essentially according to Andersson et al. (Andersson, J.; Sanchez, J.; Ekdahl, K. N.; Elgue, G.; Nilsson, B.; Larsson, R. J Biomed Mater Res A 2003, 67 (2), 458-466, incorporated herein by reference in its entirety) at 20 rpm. The platelets from fresh blood and from the blood collected from the loops are counted in a cell counter to measure the loss of platelets. A great loss of platelets indicates poor thromboresistant performance of the surface. Conversely a minimal loss of platelets indicates a thromboresistant surface.

Evaluation Method F: Blood Loop Evaluation Test (for Measurement of F1+2)

The determination of F1+2 (prothrombin fragment) is used as an activation marker for coagulation (i.e. as an indirect measurement of thrombin). F1+2 is directly proportional to the formation of thrombin and interpreted as an indirect measurement of thrombin generation, and can be used to evaluate a solid object's thromboresistant properties. Quantitative determination of F1+2 in plasma is performed with an enzymatic immunoanalysis, by using a standard ELISA kit (Enzyme-Linked Immuno Sorbent Assay) (Enzygnost F1+2 ELISA, OPBDG03, Siemens). The F1+2 antigen in the sample couples to the antibodies entrapped on the coated surface of 96-well microtiter plate and subsequently detected by a peroxidase conjugated anti-F1+2 antibody. The amount of coupled peroxidase is measured by addition of a specific substrate, 3,3′,5,5′-tetramethylbenzidine (TMB). The enzymatic conversion of the substrate to chromogen is stopped by addition of diluted sulfuric acid. Absorbance at 450 nm in the wells is proportional to the concentration of F1+2 in the sample. The concentration of the samples is determined by comparison to a standard curve with known concentrations of F1+2.

### Evaluation Method G: Molecular Weight of Anionic Polymer such as Dextran Sulfate in Solution (Molecular Weight of Anionic Polymer)

Determination of the molecular weight of a dextran sulfate sample is performed on a gel permeation chromatography (GPC) instrument. The dextran sulfate samples are dissolved in a water-based elution media and analyzed on a GPC instrument suitable for the molecular weight range 1,000 Da-100,000 Da (superose column) or 100,000 Da-2,000,000 Da (sephacryl column). A dextran sulfate standard of an appropriate molecular weight is used to verify the accuracy of the calibration curve. Polymers such as dextran sulfate are disperse molecules i.e. have a distribution of molecular weights, which can be described with different molecular weight averages. The commonly reported value is the weight average molecular weight (Mw). See Odian G., Principles of Polymerization, Third edition, Section 1.4 Molecular weight, p. 24 (incorporated herein by reference in its entirety) which explains the theory on determination of molecular weights of polymers using GPC techniques. The molecular weight of anionic polymers other than dextran sulfate can also be determined using this method.

### Evaluation Method H: Solution Charge Density of Anionic Polymer such as Dextran Sulfate in Solution (Solution Charge Density of Anionic Polymer)

Quantitative determination of charge density is performed on a Mutek Particle Charge Detector via titration of polyelectrolyte solutions (0.001 M) (polydiallyldimethylammonium chloride (Poly-Dadmac) and sodium polyethylene sulfate (PES-Na)). Samples are dissolved in water (maximum viscosity allowed 6000 mPas) to a concentration of 0.06 g/L. The pH is adjusted to 3 for all sample solutions. 10 mL per sample solution is added each measurement followed by titration of appropriate polyelectrolyte solution at an interval of 1 unit per 3 seconds. See S. Farris et al., Charge Density Quantification of Polyelectrolyte Polysaccharides by Conductometric Titration: An Analytical Chemistry Experiment, J. Chem. Educ., 2012, 89 (1), pp 121-124 (incorporated herein by reference in its entirety). The solution charge density of anionic polymers other than dextran sulfate can also be determined using this method.

### Evaluation Method I: Scanning Electron Microscopy with Energy Dispersive X-Ray Spectroscopy (Coating Coverage and Uniformity)

TM3000 is a table-scanning electron microscope (SEM) manufactured by Hitachi that is used to obtain information about e.g. a sample thickness, topography (surface structure) and surface coverage. A higher magnification can be achieved with table SEM compared to traditional light microscopes as it is electrons used to create the image. The TM3000 is also equipped with Quantax70. This is an Energy Dispersive X-ray Spectrometer (EDS) used to determine the chemical composition of the sample. In addition, there is a rotation/tilt table as accessory to facilitate analysis of different parts of the sample. The sample is mounted on a holder with carbon tape (also acts as grounding) and then placed in the test chamber. The chamber is evacuated to a lower pressure before evaluation of the sample can commence. SEM technology is based on the scanning of an electron beam across the sample, some of the electrons being reflected backscattered electrons, while others execute secondary electrons. A detector is used to measure the current generated by the reflected backscattered electrons. The current is imaged on a display where each pixel corresponds to the position of the sample. A bright pixel is obtained if many electrons are reflected (high electron density) and a darker pixel is obtained if few electrons (low electron density) are reflected.

### Evaluation Method J: X-Ray Photoelectron Spectroscopy with Depth Profiling (XPS) (Coating Thickness)

X-ray Photoelectron Spectroscopy (XPS or ESCA) is the most widely used surface characterization technique providing non-destructive chemical analysis of solid materials. Samples are irradiated with mono-energetic X-rays causing photoelectrons to be emitted from the top 1-10 nm of the sample surface. An electron energy analyzer determines the binding energy of the photoelectrons. Qualitative and quantitative analysis of all elements except hydrogen and helium is possible, at detection limits of ˜0.1-0.2 atomic percent. Analysis spot sizes range from 10 μm to 1.4 mm. It is also possible to generate surface images of features using elemental and chemical state mapping. Depth profiling is possible using angle-dependent measurements to obtain non-destructive analyses within the top 10 nm of a surface, or throughout the coating depth using destructive analysis such as ion etching.

### Evaluation Method K: Increased Temperature and Humidity Test (General Model for Sterilization Stability)

The solid object coated according to the process of the invention is placed in a breathable polyethylene pouch (e.g. a Tyvek pouch). The pouch is placed in a climate chamber (e.g. Climacell) at 40° C. and 50% relative humidity for 1 week followed by 2 hours drying in a vacuum chamber. After performing this general model for sterilization stability, the thromboresistant properties/activation of the coated object is assessed e.g. using Evaluation Method E or F.

### Evaluation Method L: Stability to Ethylene Oxide

The solid object coated according to the process of the invention is placed in a breathable polyethylene pouch (e.g. a Tyvek pouch) and subjected to at least 12 hours preconditioning at 50° C. and 60% relative humidity followed by 2 hours exposure of ethylene oxide at a pressure of 366 mBar and 50° C. The chamber is then degassed at 50° C. for at least 10 hours. Sterilization by ethylene oxide may be performed at Synergy Health Ireland Ltd. After sterilization, the thromboresistant properties/activation of the coated object is assessed e.g. using Evaluation Method E or F.

### Evaluation Method M: Heparin Activity Test (Quantitative Heparin Function using HCII)

For solid objects of the invention comprising a heparin coating, the heparin activity of the device can be measured by measuring the ability, or capacity, of the heparin to bind heparin cofactor II (HCII) as described in WO2009/064372 (Gore Enterprise Holdings, Inc.; incorporated herein by reference in its entirety) by measuring the ability, or capacity, of the heparin to bind a known quantity of heparin cofactor II (HCII), using an assay as described by Larsen M. L., et al., in “Assay of plasma heparin using thrombin and the chromogenic substrate H-D-Phe-Pip-Arg-pNA (S-2238).” Thromb Res 13:285-288 (1978) and Pasche B., et al., in “A binding of antithrombin to immobilized heparin under varying flow conditions.” Artif. Organs 1991; 15:281-491), and can be used to evaluate a solid object's thromboresistant properties. The results are expressed as picomoles heparin cofactor II (HCII) bound per apparent square centimetre of solid object surface (pmol HCII/cm² solid object surface). The apparent solid object surface area does not take into account multiple covered surfaces nor porosity considerations of a device composed of a porous material. If the surface of the device is porous, the effect of porosity on surface area is not considered for these calculations. For example, the apparent surface area of a cylindrical tubular ePTFE vascular graft (which is made of a porous material) with heparin immobilized on substrate material comprising the inner surface of the tubular graft is calculated as it is for any cylindrical geometry as 2 πrL: where r is the graft inner radius; L is the axial length; and π is the number pi. This method can be used to measure the activity of any anticoagulant entity with HCII binding activity.

### Evaluation Method N—Quartz Crystal Microbalance with Dissipation (Coating Thickness)

Q-sense E4 is a crystal microbalance with dissipation (QCM-D) monitoring instrument. QCM-D is a technique for measurement of both mass and structural properties of molecular layers and may be seen as an ultrasensitive weighing deceive.

A QCM sensor consists of a thin quartz disc where AT-cut crystals are the most commonly used. The quartz disc is placed between two electrodes and by applying a voltage to the quartz crystal it can be made to oscillate at its resonance frequency. Changes in mass on the quartz surface induces a change in frequency of the oscillating crystal related through the Sauerbrey relationship (see Rodahl, M., et al., Quartz crystal microbalance setup for frequency and Q factor measurements in gaseous and liquid environments. Review of scientific environments, 1995. 66 (7): p. 3924-3930. (incorporated herein by reference in its entirety). Coating thickness of solid objects coated according to the process of the invention are reported as dry coating thickness.

### Example 1: Processes for Coating a Solid Object (Layered Coating of Cationic and Anionic Polymer, with Outer Coating Layer of Anticoagulant Entity)

### General Coating Process—Tubing

The luminal surface of a section of tubing (e.g. PVC or PUR tubing) is coated with a layer-by-layer coating of cationic polymer and anionic polymer using essentially the method described by Larm et al. in EP0086186A1, EP0495820B1 and EP0086187A1 (all incorporated herein by reference in their entirety).

Specifically, the luminal surface of the tubing is firstly cleaned with isopropanol and an oxidizing agent. The coating bilayers are built-up by alternating adsorption of a cationic polymer (polyamine, 0.05 g/L in water) and an anionic polymer (dextran sulfate, 0.1 g/L in water). The polyamine is crosslinked with a difunctional aldehyde (crotonaldehyde). The dextran sulfate raw material is varied as specified in each of the Examples below, and applied in the presence of various sodium salts at varied concentrations, again as specified in each Example below. Every pair of polyamine and sulfated polysaccharide is called one bilayer i.e. a bilayer is defined as one layer of cationic and anionic polymer and the same conditions are used for building up of each bilayer. The luminal surface of the tubing is coated with three bilayers (see FIG. 1 for a solid object coated with a single bilayer). A final, outermost layer of polyamine is then adsorbed.

Heparin is then immobilized to the outermost layer of polyamine via reductive amination, essentially as described by Larm et al. in EP0086186A1 and EP0495820B1 (both incorporated herein by reference in their entirety).

### General Coating Process—QCM Crystals

Any solid object can be coated using the general coating process described above for tubing. In the Examples below where QCM crystals were utilized, the gold surfaces are first cleaned with ethanol, before being coated as described above for the tubing.

The complete process was carried out at a flow of 500 micro L/min. in a Q-Sense E4 system with a peristaltic pump (Ismatec IPC-N 4).

### Dextran Sulfates used in Examples 1.1-1.32

The evaluated dextran sulfates were purchased from different vendors as presented in Table 1.

TABLE 1
Dextran sulfates evaluated in the Examples

| | | | Solution charge |
|---|---|---|---|
| | | Mw | density*** |
| Dextran sulfate No. | Vendor | [kDa] | [μeq/g] (pH3) |
| 1 | Sigma Aldrich | 4* | 1.3 |
| (Reference example | | | |
| dextran sulfate) | | | |
| 2 | Sigma Aldrich | 40* | 2.9 |
| 3 | Sigma Aldrich | 50** | 6.1 |
| (Reference example | | | |
| dextran sulfate) | | | |
| 4 | Tdb Consultancy | 100** | 3.8 |
| 5 | Tdb Consultancy | 100** | 5.4 |
| (Reference example | | | |
| dextran sulfate) | | | |
| 6 | Tdb Consultancy | 600** | 3.0 |

*From vendor certificate of analysis
**Weight average molecular weight (Mw) determined according to Evaluation Method G
***Solution charge density determined according to Evaluation Method H

### Example 1.1: Preparation of Coating on PVC Tubing using Dextran Sulfate 1 and NaCl Concentration of 0.5 M

PVC tubing (I.D. 3 mm) was coated according to the general procedure described above. Dextran sulfate 1, see Table 1, was applied at NaCl concentration of 0.5 M.

### Example 1.2: Preparation of Coating on PVC Tubing using Dextran Sulfate 1 and NaCl Concentration of 1.7 M

PVC tubing (I.D. 3 mm) coated according to the general procedure described above. Dextran sulfate 1, see Table 1, was applied at NaCl concentration of 1.7 M.

### Example 1.3: Preparation of Coating on PVC Tubing using Dextran Sulfate 2 and NaCl Concentration of 0.25 M

PVC tubing (I.D. 3 mm) was coated according to the general procedure described above. Dextran sulfate 2, see Table 1, was applied at NaCl concentration of 0.25 M.

### Example 1.4: Preparation of Coating on PVC Tubing using Dextran Sulfate 2 and NaCl Concentration of 0.5 M

PVC tubing (I.D. 3 mm) was coated according to the general procedure described above. Dextran sulfate 2, see Table 1, was applied at NaCl concentration of 0.5 M.

### Example 1.5: Preparation of Coating on PVC Tubing using Dextran Sulfate 2 and NaCl Concentration of 1.7 M

PVC tubing (I.D. 3 mm) was coated according to the general procedure described above. Dextran sulfate 2, see Table 1, was applied at NaCl concentration of 1.7 M.

### Example 1.6: Preparation of Coating on Gold QCM Crystals using Dextran Sulfate 2 and NaCl Concentration of 0.05 M

Quartz Crystal Microbalance (QCM) crystals covered with gold (QSX 301, Q-Sense) were coated according to the general procedure described above. Dextran sulfate 2, see Table 1was applied at NaCl concentration of 0.05 M.

### Example 1.7: Preparation of Coating on Gold QCM Crystals using Dextran Sulfate 2) and NaCl Concentration of 0.25 M

Quartz Crystal Microbalance (QCM) crystals covered with gold (QSX 301, Q-Sense) were coated according to the general procedure described above. Dextran sulfate 2, see Table 1, was applied at NaCl concentration of 0.25 M.

### Example 1.8: Preparation of Coating on Gold QCM Crystals using Dextran Sulfate 2 and NaCl Concentration of 1.7 M

Quartz Crystal Microbalance (QCM) crystals covered with gold (QSX 301, Q-Sense) were coated according to the general procedure described above. Dextran sulfate 2, see Table 1, was applied at NaCl concentration of 1.7 M.

### Example 1.9: Preparation of Coating on Gold QCM Crystals using Dextran Sulfate 2 and NaCl Concentration of 3.4 M

Quartz Crystal Microbalance (QCM) crystals covered with gold (QSX 301, Q-Sense) were coated according to the general procedure described above. Dextran sulfate 2, see Table 1,was applied at NaCl concentration of 3.4 M.

### Example 1.10: Preparation of Coating on PVC Tubing using Dextran Sulfate 3 and NaCl Concentration of 0.25 M

PVC tubing (I.D. 3 mm) was coated according to the general procedure described above. Dextran sulfate 3, see Table 1, was applied at NaCl concentration of 0.25 M.

### Example 1.11: Preparation of Coating on PVC Tubing using Dextran Sulfate 4 and NaCl Concentration of 0.05 M

PVC tubing (I.D. 3 mm) was coated according to the general procedure described above. Dextran sulfate 4, see Table 1, was applied at NaCl concentration of 0.05 M.

### Example 1.12: Preparation of Coating on PVC Tubing using Dextran Sulfate 4 and NaCl Concentration of 0.25 M

PVC tubing (I.D. 3 mm) was coated according to the general procedure described above. Dextran sulfate 4, see Table 1, was applied at NaCl concentration of 0.25 M.

### Example 1.13: Preparation of Coating on PVC Tubing using Dextran Sulfate 4 and NaCl Concentration of 0.5 M

PVC tubing (I.D. 3 mm) was coated according to the general procedure described above. Dextran sulfate 4, see Table 1, was applied at NaCl concentration of 0.5 M.

### Example 1.14: Preparation of Coating on PVC Tubing using Dextran Sulfate 4 and NaCl Concentration of 1.7 M

PVC tubing (I.D. 3 mm) was coated according to the general procedure described above. Dextran sulfate 4, see Table 1, was applied at NaCl concentration of 1.7 M.

### Example 1.15: Preparation of Coating on PVC Tubing using Dextran Sulfate 4 and NaCl Concentration of 3.0 M

PVC tubing (I.D. 3 mm) was coated according to the general procedure described above. Dextran sulfate 4, see Table 1, was applied at NaCl concentration of 3.0 M.

### Example 1.16: Preparation of Coating on Gold QCM Crystals using Dextran Sulfate 4 and NaCl Concentration of 0.05 M

Quartz crystal microbalance (QCM) crystals covered with gold (QSX 301, Q-Sense) were coated according to the general procedure described above. Dextran sulfate 4 see Table 1, was applied at NaCl concentration of 0.05 M.

### Example 1.17: Preparation of Coating on Gold QCM Crystals using Dextran Sulfate 4 and NaCl Concentration of 0.25 M

Quartz crystal microbalance (QCM) crystals covered with gold (QSX 301, Q-Sense) were coated according to the general procedure described above. Dextran sulfate 4, see Table 1, was applied at NaCl concentration of 0.25 M.

### Example 1.18: Preparation of Coating on Gold QCM Crystals using Dextran Sulfate 4 and NaCl concentration of 1.7 M

Quartz crystal microbalance (QCM) crystals covered with gold (QSX 301, Q-Sense) were coated according to the general procedure described above. Dextran sulfate 4, see Table 1, was applied at NaCl concentration of 1.7 M.

### Example 1.19: Preparation of Coating on Gold QCM Crystals using Dextran Sulfate 4 and NaCl Concentration of 3.4 M

Quartz crystal microbalance (QCM) crystals covered with gold (QSX 301, Q-Sense) were coated according to the general procedure described above. Dextran sulfate 4, see Table 1, was applied at NaCl concentration of 3.4 M.

### Example 1.20: Preparation of Coating on PVC Tubing using Dextran Sulfate 5 and NaCl Concentration of 0.25 M

PVC tubing (I.D. 3 mm) was coated according to the general procedure described above. Dextran sulfate 5, see Table 1, was applied at NaCl concentration of 0.25 M.

### Example 1.21: Preparation of Coating on PVC Tubing using Dextran Sulfate 6 and NaCl Concentration of 0.05 M

PVC tubing (I.D. 3 mm) was coated according to the general procedure described above. Dextran sulfate 6, see Table 1, was applied at NaCl concentration of 0.05 M.

### Example 1.22: Preparation of Coating on PVC Tubing using Dextran Sulfate 6 and NaCl Concentration of 0.1 M

PVC tubing (I.D. 3 mm) was coated according to the general procedure described above. Dextran sulfate 6, see Table 1, was applied at a NaCl concentration of 0.1 M.

### Example 1.23: Preparation of Coating on PVC Tubing using Dextran Sulfate 6 and NaCl Concentration of 0.25 M

PVC tubing (I.D. 3 mm) was coated according to the general procedure described above. Dextran sulfate 6, see Table 1, was applied at NaCl concentration of 0.25 M.

### Example 1.24: Preparation of Coating on PVC Tubing using Dextran Sulfate 6 and NaCl Concentration of 1.0 M

PVC tubing (I.D. 3 mm) was coated according to the general procedure described above. Dextran sulfate 6, see Table 1, was applied at NaCl concentration of 1.0 M.

### Example 1.25: Preparation of Coating on PVC Tubing using Dextran Sulfate 6 and NaCl Concentration of 1.7 M

PVC tubing (I.D. 3 mm) was coated according to the general procedure described above. Dextran sulfate 6, see Table 1, was applied at NaCl concentration of 1.7 M.

### Example 1.26: Preparation of Coating on PVC Tubing using Dextran Sulfate 6 and NaCl Concentration of 2.6 M

PVC tubing (I.D. 3 mm) was coated according to the general procedure described above. Dextran sulfate 6, see Table 1, was applied at NaCl concentration of 2.6 M.

### Example 1.27: Preparation of Coating on PVC Tubing using Dextran Sulfate 6 and NaCl Concentration of 3.0 M

PVC tubing (I.D. 3 mm) was coated according to the general procedure described above. Dextran sulfate 6, see Table 1, was applied at NaCl concentration of 3.0 M.

### Example 1.28: Preparation of Coating on Gold QCM Crystals using Dextran Sulfate 6 and NaCl Concentration of 0.1 M

Quartz crystal microbalance (QCM) crystals covered with gold (QSX 301, Q-Sense) were coated according to the general procedure described above. Dextran sulfate 6, see Table 1, was applied at NaCl concentration of 0.1 M.

### Example 1.29: Preparation of Coating on Gold QCM Crystals using Dextran Sulfate 6 and NaCl Concentration of 0.25 M

Quartz crystal microbalance (QCM) crystals covered with gold (QSX 301, Q-Sense) were coated according to the general procedure described above. Dextran sulfate 6, see Table 1, was applied at NaCl concentration of 0.25 M.

### Example 1.30: Preparation of Coating on Gold QCM Crystals using Dextran Sulfate 6 and NaCl Concentration of 1.7 M

Quartz crystal microbalance (QCM) crystals covered with gold (QSX 301, Q-Sense) were coated according to the general procedure described above. Dextran sulfate 6, see Table 1, was applied at NaCl concentration of 1.7 M.

### Example 1.31: Preparation of Coating on Gold QCM Crystals using Dextran Sulfate 6 and NaCl Concentration of 3.0 M

Quartz crystal microbalance (QCM) crystals covered with gold (QSX 301, Q-Sense) were coated according to the general procedure described above. Dextran sulfate 6, see Table 1, was applied at NaCl concentration of 3.0 M.

### Example 1.32: Preparation of Coating on PVC Tubing using Dextran Sulfate 4 and Na₂HPO₄ Concentration of 0.85 M

PVC tubing surface was coated according to the general procedure described above. Dextran sulfate 4, see Table 1, was applied at Na₂HPO₄ concentration of 0.85 M.

### Example 2: Blood Contact Activation (Platelet Loss and F1+2) of Coated PVC Tubing using Different Dextran Sulfates at Varied NaCl Concentration

The percentage of platelets preserved and the F1+2 (prothrombin fragment) after blood exposure of PVC tubing coated according to Examples 1.3, 1.10, 1.12, 1.20 and 1.23 (corresponding to dextran sulfates 1, 2, 3, 4, 5 and 6) at varied NaCl concentration were measured as set out in Evaluation Methods E and F, respectively.

The results are shown in Table 2 (1.7 M NaCl concentration) and Table 3 (0.25 M NaCl concentration).

TABLE 2
Preserved platelets (%) and F1 + 2 (pmol/L) of PVC tubing coated
with dextran sulfates 1, 2, 4 and 6 at 1.7M NaCl concentration

| | Dextran | Preserved | | |
|---|---|---|---|---|
| | sulfate | platelets | F1 + 2 | N (number |
| Example No. | No. | [%] | [pmol/L] | average) |
| 1.2 | 1 (Ref Ex.) | 1 | 57554 | 1 |
| 1.5 | 2 | 94 | 1107 | 2 |
| 1.14 | 4 | 97 | 639 | 2 |
| 1.25 | 6 | 101 | 767 | 2 |
| Uncoated PVC | — | 1 | 637658 | — |
| example | | | | |
| Clotting | — | 1 | 644465 | — |
| example | | | | |

TABLE 3
Preserved platelets (%) and F1 + 2 (pmol/L) of PVC tubing coated
with dextran sulfates 2, 3, 4, 5, and 6 at 0.25M NaCl concentration

| | Dextran | Preserved | | |
|---|---|---|---|---|
| | sulfate | platelets | F1 + 2 | N (number |
| Example No. | No. | [%] | [pmol/L] | average) |
| 1.3 | 2 | 103 | 1138 | 2 |
| 1.10 | 3 (Ref Ex.) | 53 | 15868 | 2 |
| 1.12 | 4 | 91 | 920 | 2 |
| 1.20 | 5 (Ref Ex.) | 0 | 186405 | 1 |
| 1.23 | 6 | 91 | 920 | 2 |
| Uncoated PVC | — | 1 | 365892 | — |
| example | | | | |
| Clotting | — | 0 | 543079 | — |
| example | | | | |

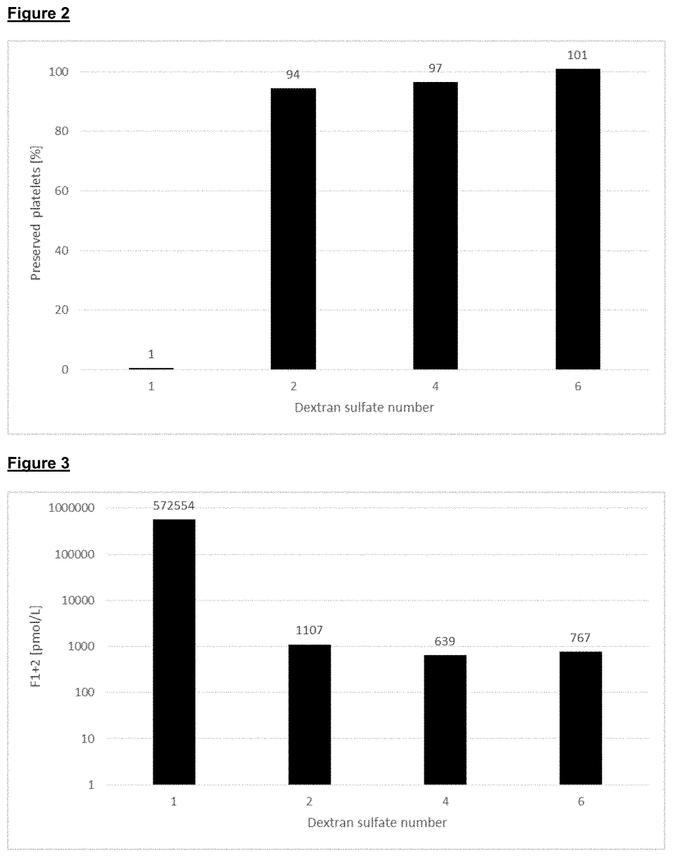

As can be seen from Table 2 and FIG. 2, no significant platelet loss (platelet loss indicating thrombosis) was observed for solid objects of the invention coated with dextran sulfates 2, 4 and 6 at 1.7 M NaCl concentration. The thromboresistant properties of the coatings were further confirmed by the low F1+2 values (low thrombin generation) observed for the same dextran sulfates, as shown in Table 2 and FIG. 3. No significant platelet loss and low F1+2 values were also observed for solid objects of the invention coated with dextran sulfates 2, 4 and 6 at a lower NaCl concentration of 0.25 M (see Table 3). The uncoated PVC tubing and the clotting example show significant thrombosis in this experiment. The tubing coated with comparative dextran sulfate 1 with molecular weight of 4 kDa also showed significant thrombosis and high thrombin generation compared with solid objects of the invention coated with dextran sulfates 2, 4 and 6 (see Table 2 and FIGS. 2 and 3).

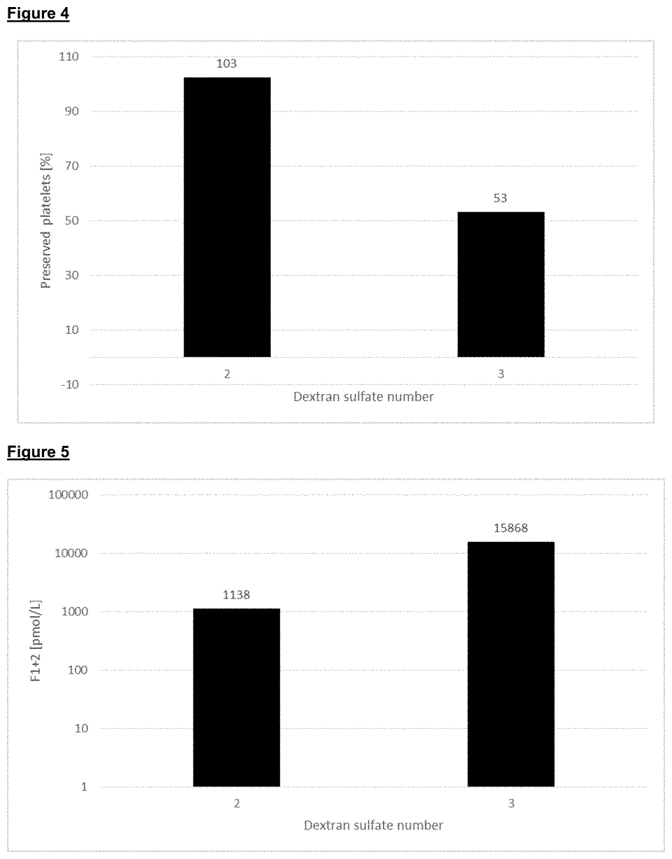

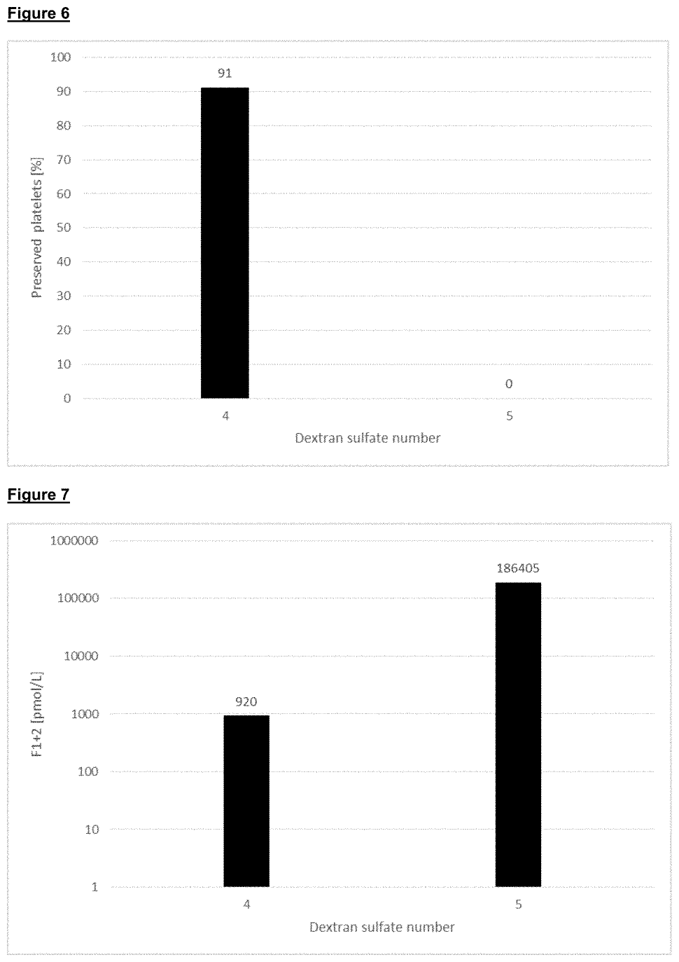

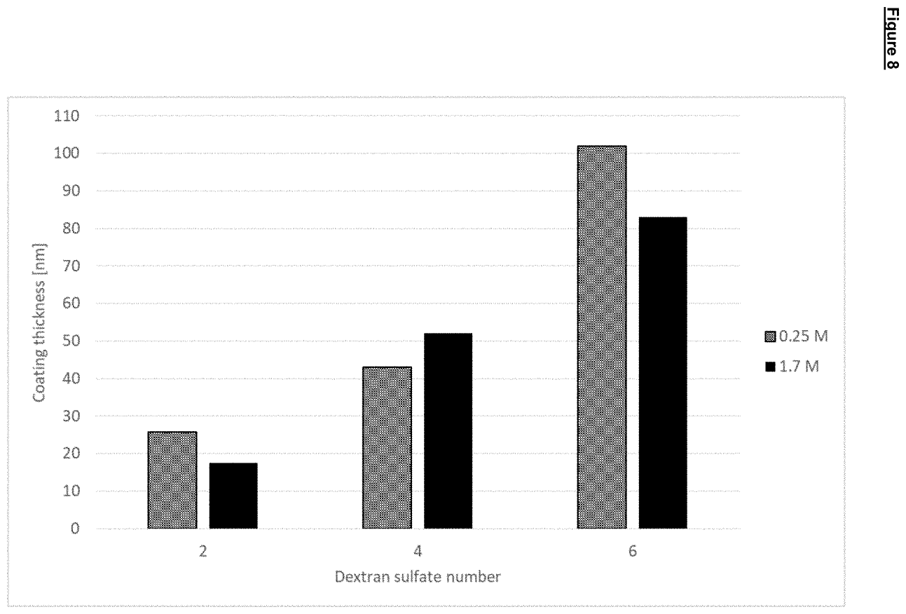

FIGS. 4 to 7 highlight the effect of the charge density of the dextran sulfate used in the layer-by-layer coating on the resulting thromboresistant properties of the final solid objects. When comparing dextran sulfates of the same/similar molecular weight coated at 0.25 M NaCl concentration, it can be seen that coatings comprising dextran sulfates with lower charge density are significantly more thromboresistant than those containing dextran sulfates with higher charge density. This is evident from FIGS. 4 and 5 where dextran sulfate 2 with lower charge density of 2.9 μeq/g exhibited significantly higher preserved platelets and lower F1+2 values compared with comparative dextran sulfate 3 with higher charge density of 6.1 μeq/g. The same trend can be seen from FIGS. 6 and 7, where dextran sulfate 4 with lower charge density of 3.8 μeq/g exhibited significantly higher preserved platelets and lower F1+2 values compared with comparative dextran sulfate 5 with higher charge density of 5.4 μeq/g.

### Example 3: Determination of Coating Thickness

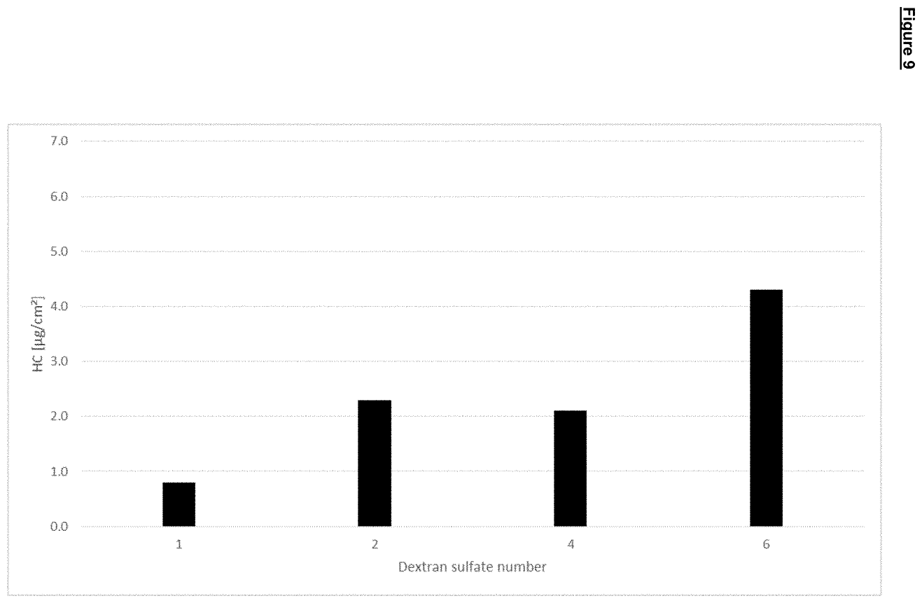

Coating thickness of PVC tubing coated according to Examples 1.6-1.1.9, 1.16-1.19 and 1.28-1.31 (corresponding to dextran sulfates 2, 4 and 6) at varied NaCl concentration was measured as set out in Evaluation Method N. The results are set out in Table 4 and FIG. 8 (0.25 M and 1.7 M NaCl concentration).

TABLE 4
Coating thickness (nm) of PVC tubing coated with dextran
sulfates 2, 4, and 6 at varied NaCl concentration

| | Dextran sulfate No. | 2 | 4 | 6 |
|---|---|---|---|---|
| Example No. | Salt concentration [M] | Coating thickness [nm] | | |
| 1.6/1.16/— | 0.05 | 22.5 | 41 | — |
| —/—/1.28 | 0.10 | — | — | 110 |
| 1.7/1.17/1.29 | 0.25 | 25.8 | 43 | 102 |
| 1.8/1.18/1.30 | 1.70 | 27.5 | 52 | 83 |
| —/—/1.31 | 3.00 | — | — | 25 |
| 1.9/1.19/— | 3.40 | 8.5 | 17 | — |

The results show the effect of the molecular weight of the dextran sulfate used in the layer-by-layer coating on the overall coating thickness.

The coating thickness increases with increasing molecular weight, shown in Table 4 and graphically in FIG. 8, where it can be seen that tubing coated with dextran sulfate 6 (600 kDa) had a had a thicker coating than tubing coated with dextran sulfate 4 (100 kDa), which in turn had a thicker coating that tubing coated with dextran sulfate 2 (40 kDa).

Whether a relatively thicker or thinner coating is desired depends on the intended application of the solid object. Being able to modify the coating thickness is therefore advantageous. It can be seen from FIG. 8 that coating thickness of the final solid object is to a certain extent also dependent on the salt concentration used when applying the dextran sulfate layer(s). For dextran sulfates 2 and 6 with charge density of 2.9 μeq/g and 3.0 μeq/g, respectively, it was found that using a lower NaCl concentration of 0.25 M compared with 1.7 M resulted in a thicker coating. For dextran sulfate 4 with slightly higher charge density of 3.8 μeq/g the opposite was observed, where using a higher NaCl concentration of 1.7 M resulted in a thicker coating.

### Example 4: Heparin Concentration of Coated PVC Tubing using Different Dextran Sulfates at Varied NaCl Concentration

Heparin concentration of solid objects (PVC tubing) coated according to Examples 1.1-1.5, 1.11-1.15 and 1.21-1.27 (corresponding to dextran sulfates 1, 2, 4 and 6) at varying NaCl concentrations was measured as set out in Evaluation Method A.

Results are shown in Table 5.

TABLE 5
Heparin concentration (μg/cm²) of coated PVC tubing with dextran
sulfates 1, 2, 4 and 6 at varied NaCl concentration

| | Dextran | | | | |
|---|---|---|---|---|---|
| | sulfate No. | | | | |
| | Salt | | | | |
| | concentration | 1 | 2 | 4 | 6 |
| Example No. | [M] | Heparin concentration [μq/cm²] | | | |
| —/—/1.11/1.21 | 0.05 | — | — | 2.3 | 4.8 |
| —/—/—/1.22 | 0.10 | — | — | — | 4.8 |
| —/1.3/1.12/1.23 | 0.25 | — | 1.7 | 2.1 | 5.1 |
| 1.1/1.4/1.13/— | 0.50 | 0.8 | 2.1 | 2.2 | — |
| —/—/—/1.24 | 1.00 | — | — | — | 5.1 |
| 1.2/1.5/1.14/1.25 | 1.70 | 0.8 | 2.3 | 2.1 | 4.3 |
| —/—/—/1.26 | 2.60 | — | — | — | 3.1 |
| —/—/1.15/1.27 | 3.00 | — | — | 1.5 | 2.0 |

As can be seen from Table 5 and FIG. 9, solid objects of the invention coated with dextran sulfates 2, 4 and 6 at 1.7 M NaCl concentration exhibited heparin concentration of greater than 1 μg/cm². Tubing coated with comparative dextran sulfate 1 with molecular weight of 4 kDa exhibited a lower heparin concentration of 0.8 μg/cm².

It is evident from Table 5 and FIG. 9 that the molecular weight of the dextran sulfate used in the layer-by-layer coating has an effect on the heparin concentration of the final solid object, where the heparin concentration increases with the molecular weight of the dextran sulfate. This can be seen by comparing dextran sulfates 2 and 6 of similar charge density (2.9 μeq/g and 3.0 μeq/g respectively) coated at 1.7 M NaCl concentration, where dextran sulfate 6 (600 kDa) exhibited a higher heparin concentration than dextran sulfate 2 (40 kDa).

It can be seen from Table 5 that heparin concentration of the final solid object of the invention is to a certain extent also dependent on the salt concentration used when applying the dextran sulfate layer(s). See for example dextran sulfate 6, where the highest heparin concentration was observed at NaCl concentrations of 0.25 M, whereas higher salt concentrations resulted in a lower heparin concentration. The salt concentration when applying the dextran sulfate layer(s) can therefore be varied to optimize the heparin concentration for a particular dextran sulfate.

Preparation of coatings using sodium hydrogen phosphate (Na₂HPO₄) and sodium chloride (NaCl) as salt when applying the dextran sulfate layer was performed according to Example 1.13, 1.14 and 1.32. Results show that the use of alternative salts such as for example Na₂HPO₄ when applying dextran sulfates according to the present invention resulted in solid objects which exhibited heparin concentration of greater than 1 μg/cm². Surfaces coated with dextran sulfate 4 using Na₂HPO₄ obtained a heparin concentration of 1.7 μg/cm² at 0.85 M Na₂HPO₄ compared to 2.2 μg/cm² at 0.5 M and 2.1 μg/cm² at 1.7 M NaCl.

### Example 5: Toluidine Blue Staining of Coated PVC Tubing using Different Dextran Sulfates at Varied Salt Concentration

PVC tubing coated according to Examples 1.1-1.32 were subjected to a toluidine blue staining test as set out in Evaluation Method C.

A blue/violet color was observed on the luminal surface of the tubing indicating the covalent attachment of end-point functionalized heparin. The homogenous staining obtained for tested tubing indicates formation of a uniform coating (in particular uniform heparin distribution) which may be obtained using different dextran sulfates at different salt concentrations.

### Example 6: Blood Contact Activation (Platelet Loss and F1+2) of Coated PVC Tubing—Post Temperature and Humidity Test

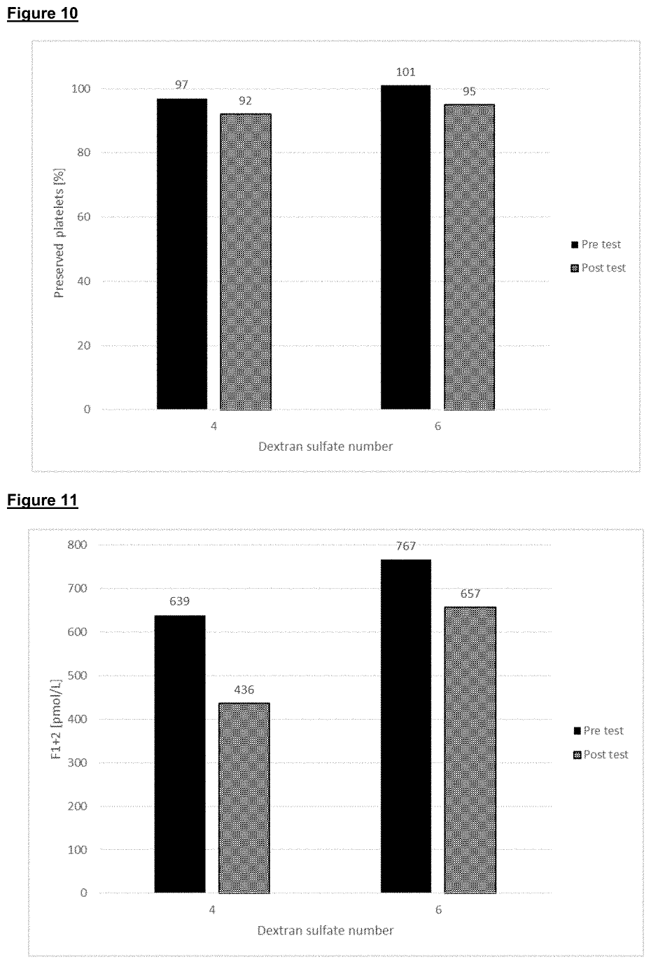

PVC tubing coated according to Examples 1.14 and 1.25 (corresponding to dextran sulfates 4 and 6) at 1.7 M NaCl concentration were exposed to increased temperature and relative humidity (40° C., 50% RH, 1 week) prior to evaluation according to Evaluation Methods E (preserved platelets) and F (F1+2). The results are shown in Table 6 and FIGS. 10 (preserved platelets) and 11 (F1+2).

TABLE 6
Preserved platelets (%) and F1 + 2 (pmol/L) of PVC tubing coated
with dextran sulfate 4 and 6 at 1.7M NaCl concentration-before and
after exposure to increased temperature and humidity

| Exposure to | | Dextran | Preserved | | |
|---|---|---|---|---|---|
| 40° C. 50% RH, | Example | sulfate | platelets | F1 + 2 | N (number |
| 1 week | No. | No. | [%] | [pmol/L] | average) |
| Pre | 1.14 | 4 | 97 | 639 | 2 |
| Post | | 4 | 92 | 436 | 1 |
| Pre | 1.25 | 6 | 101 | 767 | 2 |
| Post | | 6 | 95 | 657 | 1 |
| Pre | Uncoated | — | 0 | 285739 | — |
| Post | PVC | | 1 | 619778 | — |
| | example | | | | |
| Pre | Clotting | — | 0 | 698188 | — |
| Post | example | | 2 | 768361 | — |

As seen in the Tables and Figures, for solid objects of the invention coated with dextran sulfates 4 (40 kDa) and 6 (600 kDa), there is very little change in the preserved platelet values post exposure to increased temperature and humidity. F1+2 values were lower (indicated lower thrombin generation) post exposure to temperature and humidity. These results demonstrate that the thromboresistant properties of the coated solid objects prepared according to the invention are retained in spite of exposure to rigorous conditions as increased temperature and humidity.

All patents and patent applications referred to herein are incorporated by reference in their entirety.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

1. A solid object having a surface comprising a layered coating of cationic and anionic polymer, wherein the outer coating layer is a layer comprising cationic polymer to which is covalently bound an anticoagulant entity;
and wherein the anionic polymer is characterized by having (a) a total molecular weight of 20 kDa-650 kDa; and (b) a solution charge density of ≤4 μeq/g.

2. A solid object according to claim 1 obtainable by a process comprising the steps of:
i) treating a surface of the solid object with a cationic polymer;
ii) treating the surface with an anionic polymer;
iii) optionally repeating steps i) and ii) one or more times;
iv) treating the surface with a cationic polymer; and
v) treating the outermost layer of cationic polymer with an anticoagulant entity, thereby to covalently attach the anticoagulant entity to the outermost layer of cationic polymer;
wherein, the anionic polymer is characterized by having (a) a total molecular weight of 20 kDa-650 kDa; and (b) a solution charge density of ≤4 μeq/g.

3. A process for the manufacture of a solid object having a surface comprising a layered coating of cationic and anionic polymer, wherein the outer coating layer comprises cationic polymer to which is covalently bound an anticoagulant entity, comprising the steps of:
i) treating a surface of the solid object with a cationic polymer;
ii) treating the surface with an anionic polymer;
iii) optionally repeating steps i) and ii) one or more times;
iv) treating the surface with a cationic polymer; and
v) treating the outermost layer of cationic polymer with an anticoagulant entity, thereby to covalently attach the anticoagulant entity to the outermost layer of cationic polymer;
wherein, the anionic polymer is characterized by having (a) a total molecular weight of 20 kDa-650 kDa; and (b) a solution charge density of ≤4 μeq/g.

4. A solid object according to claim 1, wherein the anionic polymer is dextran sulfate.

5. A solid object according to claim 1, wherein the anionic polymer is characterized by having a total molecular weight of 20 kDa-125 kDa.

6. A solid object according to claim 1, wherein the anionic polymer is characterized by having a total molecular weight of 20 kDa-75 kDa.

7. A solid object according to claim 1, wherein the anionic polymer is characterized by having a total molecular weight of 75 kDa-125 kDa.

8. A solid object according to claim 1, wherein the anionic polymer is characterized by having a total molecular weight of 525 kDa-650 kDa.

9. (canceled)

10. (canceled)

11. A solid object according to claim 1, wherein the anionic polymer is applied to the surface at a salt concentration of 0.05 M-3.0 M.

12. (canceled)

13. (canceled)

14. A solid object according to claim 11, wherein the salt is an inorganic sodium salt.

15. (canceled)

16. (canceled)

17. (canceled)

18. (canceled)

19. A solid object according to claim 1, wherein the cationic polymer is a polyamine.

20. (canceled)

21. A process for the manufacture of a solid object according to claim 3, wherein the cationic polymer of step i) is a polyamine, which is optionally cross-linked.

22. A process for the manufacture of a solid object according to claim 3, wherein the cationic polymer of step iv) is a polyamine.

23. (canceled)

24. (canceled)

25. A solid object according to claim 1, wherein the anticoagulant entity is a heparin moiety.

26. A solid object according to claim 25, wherein the heparin moiety is an end-point attached heparin moiety.

27. A solid object according to claim 26, wherein the end-point attached heparin moiety is connected through its reducing end.

28. A solid object according to claim 27, wherein the anticoagulant entity is a full length heparin.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. A solid object according to claim 1, with coating thickness of ≤300 nm e.g. ≤250 nm, e.g. ≤200 nm, ≤150 nm, ≤100 nm, ≤75 nm, ≤50 nm, ≤40 nm, ≤30 nm or ≤25 nm.

44. A solid object having a surface comprising a layered coating of cationic and anionic polymer, wherein the outer coating layer is a layer comprising cationic polymer;
and wherein the anionic polymer is characterized by having (a) a total molecular weight of 20 kDa-650 kDa; and (b) a solution charge density of ≤4 μeq/g.

45. (canceled)

46. (canceled)

47. A solid object having a surface comprising a layered coating of cationic and anionic polymer, wherein the outer coating layer is a layer comprising anionic polymer; and wherein the anionic polymer is characterized by having (a) a total molecular weight of 20 kDa-650 kDa; and (b) a solution charge density of ≤4 μeq/g.

48. (canceled)

49. (canceled)
